(19) United States
(12) Patent Application Publication
(10) Pub. No.: US 2015/0224120 A1
(43) Pub. Date: Aug. 13, 2015

(54) COMPOSITIONS AND METHODS FOR TREATING HYPERPROLINEMIA-ASSOCIATED MENTAL DISORDERS

(71) Applicants: Catherine Clelland, New York, NY (US); James Clelland, New York, NY (US)

(72) Inventors: Catherine Clelland, New York, NY (US); James Clelland, New York, NY (US)

(21) Appl. No.: 14/344,751

(22) PCT Filed: Sep. 14, 2012
(86) PCT No.: PCT/US2012/055523

§ 371 Date: Feb. 11, 2015

Related U.S. Application Data
(60) Provisional application No. 61/534,688, filed on Sep. 14, 2011.

(51) Int. Cl. A61K 31/593; G01N 33/573; A61K 45/06; A61K 31/5513; A61K 31/12; A61K 31/4439

(52) U.S. Cl. CPC A61K 31/593; A61K 31/12; A61K 31/4439; A61K 45/06; A61K 31/5513; G01N 33/573; G01N 2333/9065; G01N 2800/302

(57) ABSTRACT

The present invention provides methods for treating or ameliorating the effects of schizophrenia, which include administering to a patient in need thereof a therapeutically effective amount of a proline modulator. Further provided are methods of selecting a patient at risk for or suffering from schizophrenia that is likely to benefit from proline modulation. Methods for identifying an agent that modulates proline levels in a patient and methods for identifying a patient at risk for developing a DTNBP1-related psychiatric illness, as well as other methods and compositions for treating or ameliorating the effects of schizophrenia are also provided.

### CROSS-REFERENCE TO RELATED APPLICATIONS

The present invention claims benefit to U.S. provisional application Ser. No. 61/534,688 filed Sep. 14, 2011, the entire contents of which are incorporated by reference.

### GOVERNMENT FUNDING

This invention was made with government support under grant no. MH070601-02 awarded by the National Institute of Mental Health (NIMH) and grant nos. KL2 RR024157 and 1UL1 RR029893 awarded by the National Center for Research Resources. The government has certain rights in the invention.

### FIELD OF INVENTION

The present invention provides, inter alia, compositions and methods for treating hyperprolinemia-associated mental disorders, such as e.g., schizophrenia.

### BACKGROUND OF THE INVENTION

Schizophrenia is a severe psychiatric disorder of unknown cause, with a worldwide incidence of approximately 1%. There is a large increased risk of schizophrenia and other psychotic disorders in people with 22q11 deletion syndrome (22q11 DS), with up to one third developing schizophrenia or schizoaffective disorder (Murphy et al., 1999; Scambler, 2000; Jacquet et al., 2002; Karayiorgou and Gogos, 2004; Karayiorgou et al., 2010). A common feature of 22q11 DS is a hemizygous deletion of the proline dehydrogenase (PRODH) gene, which encodes the proline dehydrogenase enzyme, that catalyzes the first step in proline catabolism (Mitsubuchi et al., 2008). Significantly, approximately 37-50% of patients with the 22q11 deletion (Goodman et al., 2000; Raux et al., 2007) have elevation of plasma proline that is between 2 and 10 fold higher than the upper end of the normal range (Mitsubuchi et al., 2008), and plasma proline levels have been found to inversely correlate with intelligence quotient in patients with the 22q11 DS velo-cardiofacial syndrome (Raux et al., 2007).

In addition to its proteogenic role, proline is a precursor of the neurotransmitter glutamate, and has several characteristics that suggest it functions as a CNS neuromodulator (Phang et al., 2001). Studies of elevated proline in humans and model systems illustrate some of the pathogenic properties of hyperprolinemia. In the hyperprolinemic PRO/RE mouse strain, elevated peripheral and CNS proline are associated with neurocognitive dysfunction, in the form of learning and memory deficits (Baxter et al., 1985; Davis et al., 1987). Deficiency of PRODH activity in the PRO/RE mouse, which results from a heterozygous nonsense Prodh mutation (the premature termination E453X variant (Gogos et al., 1999)), closely mimics the loss of PRODH activity and the 2-10 fold elevation of plasma proline observed in human hyperprolinemia type-I (HPI), which also arises from mutations in the PRODH gene (Mitsubuchi et al., 2008). Although variable, the neurological phenotype associated with HPI includes mental retardation and epilepsy (Afenjar et al., 2007; Mitsubuchi et al., 2008). Plasma proline elevations greater than 10-fold above the normal range are found in patients with hyperprolinemia type-II (HPII), caused by mutations in the ALDH4A1 gene that encodes Δ-1-pyrroline-5-carboxylate (P5C) dehydrogenase, which is immediately downstream of PRODH in proline catabolism. P5C dehydrogenase deficits and the resultant hyperprolinemia can lead to low IQ, seizures, and in some subjects, mild mental retardation (Flynn et al., 1989).

Following chronic proline administration, rats with plasma proline levels consistent with human HPII developed behavioral and brain histological changes coupled with impairments of glutamate synthesis, all suggestive of neurological dysfunction (Shanti et al., 2004).

Evidence supporting the functional significance of hyperprolinemia in schizophrenia comes from two sources: mice homozygous for the Prodh E453X mutation, have elevated plasma and brain proline, locally decreased CNS glutamate and γ-aminobutyric acid (GABA) (Gogos et al., 1999; Paterlini et al., 2005), and a deficit in sensorimotor gating shown as decreased prepulse inhibition of the acoustic startle response, that is a characteristic of schizophrenia (Braff et al., 1978). Moreover, familial PRODH deletion and PRODH missense mutations that have been described in patients with schizophrenia (Jacquet et al., 2002; Bender et al., 2005), and that have been functionally related to both moderately and severely decreased PRODH enzyme activity in vitro (Bender et al., 2005), have also been associated with both HPI and moderate hyperprolinemia in schizophrenic patients (Jacquet et al., 2002). However, the conclusions of case-control studies evaluating peripheral proline levels as a risk factor for schizophrenia have been conflicting. Following measurement of plasma proline, Jacquet et al. did not detect an association between mild to moderate hyperprolinemia and schizophrenia in a mixed-gender study of Caucasian subjects, although they did report hyperprolinemia as a significant risk for schizoaffective disorder (Jacquet et al., 2005). This study concurred with a previous report, finding no significant difference in serum proline level across groups of control subjects, treated schizophrenics, naive schizophrenics and drug-free schizophrenic subjects (Rao et al., 1990). Conversely, a more recent study also measuring serum levels found a significant elevation of proline in schizophrenic patients when compared to controls, but only in female subjects (Tomiya et al., 2007). Despite these mixed findings, data continues to support a functional role for PRODH variants and hyperprolinemia in the etiology of schizophrenia (Kempf et al., 2008), although studies relating plasma proline level to the clinical symptoms of schizophrenia are lacking.

SZ is a common disorder with a large genetic component. SZ is a severe and debilitating psychiatric disorder of unknown cause, with a worldwide incidence of approximately 1%. Susceptibility to SZ has large genetic and heritable components, indicated in studies of increased risk among first degree relatives, and of concordance between mono- and dizygotic twins 20-22. Individuals with SZ display a wide range of symptoms suggesting underlying physical, biological and/or environmental etiological differences between individuals.

1α,25(OH)₂D₃ (25-hydroxyvitamin-D, the sum of 25-hydroxyvitamin D₃ and 25-Hydroxyvitamin D₂) is a pleotropic steroid hormone that is primarily synthesized in the skin from the enzymatic conversion of 7-dehydrocholesterol to the active form in the presence of sunlight emitted ultraviolet B (UVB) light, as well as derived from some food sources. 25-hydroxyvitamin-D has a well-established and vital role in the maintenance of calcium homeostasis and bone mineral density. However, 25-hydroxyvitamin-D also regulates transcription of a large number of genes, directly or indirectly influencing cell cycling and proliferation, differentiation, and apoptosis. And, insufficiency or deficiency of 25-hydroxyvitamin-D, defined as serum or plasma levels of ≦30 ng/ml and <20 ng/ml respectively, have been associated with metabolic, immune, and malignant disease (reviewed in Rosen, 2011).

Data from epidemiological studies has also implicated Vitamin-D deficits in the risk for psychiatric illness, in particular, susceptibility to schizophrenia (McGrath, 2010). Specifically, environmental factors such as prenatal nutrition deficiency, winter/spring birth, birth in an urbanized area or at high latitude, and migrant status, particularly migrants with dark skin tones migrating to colder climates, have all been associated with increased schizophrenia risk, and all potentially share the underlying factor of Vitamin-D deficiency, due to decreased skin exposure to UVB light and/or poor diet. Biological plausibility for this as a schizophrenia risk factor comes from a rat model of maternal developmental vitamin-D deficiency (DVD), neonates from which exhibit increased cellular proliferation, reduced apoptosis, altered neurogenesis, and disturbances in dopamine ontology, while adult animals had enhanced locomotion when exposed to amphetamine, as well as to an NMDA receptor antagonist, the characteristics of human schizophrenia.

Although multiple hypotheses exists, the mechanism by which a Vitamin D deficit may confer schizophrenia risk is currently unknown. Nevertheless, direct evidence for the association with increased schizophrenia risk comes from two important infant cohort studies. Firstly, from analysis of a large birth cohort, McGrath et al. (2000) reported a significantly reduced risk of schizophrenia in male infants receiving vitamin-D supplements (≦2000 IU/day) during their first year of life. Retrospective measurement of 25-hydroxyvitamin-D₃ in neonatal dried blood spots from over 800 schizophrenia patients and matched controls, then demonstrated that infants with low Vitamin-D had a significantly increased risk of schizophrenia. Consistent with these finding, higher 25-hydroxyvitamin-D₃ has been associated with a lower risk of psychotic experiences in children. Taken together, this body of work has led to the recommendation of maternal, neonatal, infantile or early childhood Vitamin-D supplementation for those at-risk.

Interestingly, a number of small studies have more recently suggested that 25-hydroxyvitamin-D insufficiency in schizophrenia extends into adulthood: Significantly lower serum Vitamin-D levels have been reported in adult inpatients with chronic schizophrenia compared to other psychiatric inpatients, and healthy controls (Humble et al., 2010). The importance of Vitamin-D level maintenance in the adult psychiatric population has also been highlighted by a large cohort study, from which it was reported that women with high dietary Vitamin-D consumption had a 37% lower risk of psychosis-like symptoms compared to women with low consumption.

There is a large increased risk of SZ and other psychotic disorders in people with 22q11 DS, with up to one third developing SZ or schizoaffective disorder (SaD) 4; 23-26. A common feature of 22q11DS is a hemizygous deletion of the proline dehydrogenase (PRODH) gene, which encodes the proline dehydrogenase enzyme, that catalyses the first step in proline catabolism 2. Significantly, approximately 37-50% of patients with the 22q11 deletion 27; 28 have elevation of plasma proline that is between 2-10 fold higher than the upper end of the normal range 2, and plasma proline levels have been found to inversely correlate with intelligence quotient in patients with the 22q11 DS velo-cardiofacial syndrome.

In addition to its proteogenic role, proline is a precursor of the neurotransmitter glutamate, and has several characteristics that suggest it functions as a CNS neuromodulator. Proline has several properties that are similar to classical excitatory amino acid neurotransmitters, such as its release at the synapse after K⁺-induced depolarization, its synthesis within synaptosomes and its uptake into synaptosomes by a high-affinity Na-dependent transport system. In addition, the PROT high affinity proline transporter is differentially expressed in a subpopulation of excitatory nerve terminals and proline can modulate glutamatergic neurotransmission, further supporting a CNS neurotransmission-related role for proline.

Studies of elevated proline in humans and model systems illustrate some of the pathogenic properties of hyperprolinemia: in the hyperprolinemic PRO/RE mouse strain, elevated peripheral and CNS proline is associated with neurocognitive dysfunction in the form of learning and memory deficits. Deficiency of PRODH activity in the PRO/RE mouse, which results from a heterozygous nonsense Prodh mutation (the premature termination E453X5), closely mimics the loss of PRODH activity and the 2-10 fold elevation of plasma proline observed in human hyperprolinemia type-I (HPI), which also arises from mutations in PRODH2. Although variable, the neurological phenotype associated with HPI includes mental retardation and epilepsy. Plasma proline elevations greater than 10-fold above the normal range are found in patients with hyperprolinemia type-II (HPII), caused by mutations in the ALDH4A1 gene that encodes Δ-1-pyrroline-5-carboxylate (P5C) dehydrogenase, which is immediately downstream of PRODH in proline catabolism. P5C dehydrogenase deficits and the resultant hyperprolinemia can lead to low IQ, seizures, and in some subjects, mild mental retardation. Following chronic proline administration, rats with plasma proline levels consistent with human HPII developed behavioral and brain histological changes coupled with impairments of glutamate synthesis, all suggestive of neurological dysfunction.

Evidence supporting the functional significance of hyperprolinemia in SZ comes from two sources: mice homozygous for the Prodh E453X mutation, have elevated plasma and brain proline, locally decreased CNS glutamate and γ-aminobutyric acid (GABA) and a deficit in sensorimotor gating shown as decreased prepulse inhibition of the acoustic startle response, that is a characteristic of SZ 44. Moreover, familial PRODH deletion and PRODH missense mutations that have been described in patients with SZ, and that have been functionally related to both moderately and severely decreased PRODH enzyme activity in vitro, have also been associated with both HPI and moderate hyperprolinemia in SZ patients.

The conclusions of case-control studies evaluating peripheral proline levels as a risk factor for SZ have been conflicting: following measurement of plasma proline, Jacquet et al. did not detect an association between mild to moderate hyperprolinemia and SZ in a mixed-gender study of Caucasian subjects, although they did report hyperprolinemia as a significant risk for schizoaffective disorder. Conversely, a more recent study measuring serum levels found a significant elevation of proline in SZ patients when compared to controls, but which was only significantly higher in female subjects, although the small sample size they employed likely contributed to the insignificant finding in males.

Association studies of PRODH and SZ are also discordant. In 2002, one of the first case-control association studies, performed due to the location of PRODH on 22q11 and its proximity to the critical region deletion breakpoints, reported a significant association to a PRODH 3′ SNP with SZ. Although this study was replicated in at least two different and distinct populations other studies, two of which used the same marker sets as the original report and greater subject numbers, plus one which employed a dense marker set determined from sequencing in entirety the PRODH coding region 48, have since failed to replicate the association with SZ. Of relevance, and despite these mixed findings, data continues to support a functional role for PRODH variants and hyperprolinemia in the etiology of SZ.

Analysis of PRODH transcription may augment DNA analysis. It has been suggested that analysis at the level of PRODH transcription may also be beneficial in understanding the role of PRODH in the etiology of hyperprolinemia/SZ. As mentioned above, Bender et al., performed a rigorous and detailed study, determining the functional consequences (at the level of in vitro PDX activity), of PRODH missense mutations, three of which were previously identified in SZ and were shown to severely reduce activity. However, it is noted that variants with limited in vitro loss of activity, may have profound in vivo effects due to the alteration of, e.g. splicing and/or mRNA stability.

Current treatment strategies do not treat all SZ symptoms, and can lead to non-compliance. Long-term treatment of SZ is necessary to maintain the alleviation of symptoms that include positive (delusions, hallucinations) cognitive (disorganized speech, memory problems), and negative symptoms (apathy, grossly disorganized or catatonic behavior, lack of emotion, poor or nonexistent social functioning). Current treatments include use of low dose first generation “typical” antipsychotics and/or higher doses of the second generation ‘atypicals’. Randomized clinical trials have shown that atypicals have similar efficacy in alleviating SZ symptoms, but significantly less of the typical neuroleptic-associated side-effects. However, studies have also shown that the atypical neuroleptic drug efficacy positively correlates with other metabolic disturbances such as hyperglycemia, hyperlipidemia, extensive weight gain and diabetes, all of which can decrease patient compliance to the required long-term treatment regime. In addition, it has been reported that as many as 30% of all SZ subjects do not respond to currently available treatments and upward of 60% have partial response with residual symptoms persisting. Moreover, no medications are currently approved for treatment of residual psychotic, negative or cognitive symptoms in SZ.

There is a continuing need for new therapeutic approaches for treatment of SZ. As noted above, both first and second generation antipsychotics have significant associated side-effects, up to 30% of SZ sufferers do not respond to currently available treatments, and residual symptoms remain in over 60% of SZ patients. It seems clear that new therapies must be developed, that are associated with a rapid improvement in active psychotic symptomatology in all SZ patients, and also increase patient tolerance and thus long-term adherence. The present invention is directed to meeting these and other needs.

### SUMMARY OF THE INVENTION

One embodiment of the present invention is a method for treating or ameliorating the effects of a schizophrenia-spectrum disorder. This method comprises administering to a patient in need thereof a therapeutically effective amount of a proline modulator.

Another embodiment of the present inventions is a method of selecting a patient at risk for or suffering from schizophrenia likely to benefit from proline modulation. This method comprises:
(a) obtaining a biological sample from the patient;
(b) testing the biological sample to determine whether the patient has hyperprolinemia, wherein a patient with hyperprolinemia is a candidate for proline modulation treatment; and
(c) if the patient is determined from step (b) to have hyperprolinemia, administering to the patient an effective amount of an activator of PRODH or an activator of PPARγ.

Yet another embodiment of the present invention is a composition for treating or ameliorating the effects of schizophrenia. This composition comprises an effective amount of a proline modulator, and a pharmaceutically acceptable carrier.

A further embodiment of the present invention is a method for identifying an agent that modulates proline levels in a patient. This method comprises:
(a) administering a candidate agent to a non-human animal having a null mutation of Dtnbp 1;
(b) carrying out an assay to determine whether the candidate agent changes the proline level or the PRODH level in the non-human animal relative to a control;
   wherein a candidate agent that causes a change in the proline level or the PRODH level of the non-human animal relative to the control is an agent that modulates proline levels in a patient.

Another embodiment of the present invention is a method for identifying whether a patient is at risk for developing a DTNBP1-related psychiatric illness, or whether a patient having a schizophrenia-spectrum disorder is at risk for an increased length of hospital stay. This method comprises:
(a) obtaining a biological sample from a patient;
(b) carrying out an assay to determine whether the patient has an elevated proline level compared to a control (proline assay) or a decreased PRODH expression level relative to a control (PRODH assay),
   wherein a patient with an elevated proline level or a decreased PRODH expression level in step (b) is at risk for developing a DTNBP1-related psychiatric disease and/or is at risk for an increased length of hospital stay.

### BRIEF DESCRIPTION OF THE DRAWINGS

FIG. 1 shows hyperprolinemia in schizophrenia (SZ), and the effect on age at first hospitalization (AFH) and length of hospital stay (LOHS). FIG. 1A shows fasting plasma proline in SZ and control groups. The box plot illustrates the significant difference between control (174.28±55.97) and SZ patient (215.84±63.00) groups, Mann-Whitney z=−4.58, p<0.0001. FIG. 1B shows a bivariate relationship between AFH and hyperprolinemia. Hyperprolinemic SZ patients have a significantly later age of first hospitalization (29.9±10.2 years) compared to non-hyperprolinemic patients (22.2±5.4 years), log-normal model: z=3.37, 1df, p=0.001. Age at first hospitalization could not be determined for 17 subjects. FIG. 1C shows a bivariate relationship between LOHS and hyperprolinemia. The duration of their hospital stay is longer for hyperprolinemic SZ patients (47.0±19.7 days), compared to non-hyperprolinemic patients (30.1±21.9 days), gamma-log model: z=2.38, 1df, p=0.017. 19 subjects were excluded from analysis because they were transferred or discharged to another treatment facility. A gamma-log model showed no effect of AFH on LOHS for all 35 subjects for whom AFH could be determined and who were not transferred to a second care facility (z=1.35, 1df, p=0.178), or for the subset of 9 hyperprolinemic subjects (z=1.04, 1df, p=0.298). Key: SZ: Schizophrenia, NH: Non-hyperprolinemic, H: Hyperprolinemic, AFH: Age at first hospitalization, LOHS: length of hospital stay, IQR: interquartile range. The jittered points represent individual subject data. The horizontal line within each box represents the group mean (mean±SD reported). The box indicates the IQR. The whiskers extend to the most extreme data point which is <1.5 times the IQR.

FIG. 2 shows fasting plasma proline in control, bipolar disorder (BPD) and schizophrenia (SZ) groups. The box plot illustrates that schizophrenia patients (n=64) had significantly higher proline compared to a matched cohort of BPD patients (n=40) and controls (n=90), p<0.0001. Categorical analysis of gender adjusted hyperprolinemia showed a significant association with schizophrenia, but not BPD. Key: Gender adjusted Proline Z-score=(subject proline level−mean of gender-specific control group)/SD of gender specific control group. The jittered points represent individual subject data. The horizontal line within each box represents the gender adjusted proline group mean. The box indicates the interquartile range. The whiskers represent 25th and 75th percentiles of the data.

FIG. 3 shows the responses of PRODH expression/PDX enzyme activation to various stimuli and their metabolic consequences. The circle highlights targeted pathways of relevance to the subject matter of the present invention. Key: ATP, adenosine triphosphate; COX2, cyclooxygenase-2; ETC, electron transport chain; GADD, growth arrest DNA damage; GLU, glutamic acid; HIF-1α, hypoxia-inducible factor-1α; NAD, nicotinamide adenine dinucleotide; NADP, nicotinamide adenine dinucleotide phosphate; PPARγ, peroxisomal proliferator-activated receptor gamma; PDX, proline oxidase; PRO, proline; Pyr, pyruvate; ROS, reactive oxygen species.

FIG. 4 shows the effect of vitamin D treatment in various systems. FIG. 4A shows the effect of Vitamin D on intestinal epithelial cell PRODH expression (probeset 40042_r_at shown). PRODH was upregulated in the Vitamin D treated epithelial cells (n=5), when compared to untreated control cells (n=5), p=0.044 (one-tailed). FIG. 4B shows the effect of vitamin D on PRODH expression from bronchial smooth muscle cells (probeset 214203_s_at shown). A similar upregulation of PRODH expression was observed in treated (n=3), versus untreated cells (n=3), p=0.01. Affymetrix microarray data was accessed via the NCBI GEO database, accession numbers GDS1847 (FIG. 4A) and GDS2628 (FIG. 4B). FIG. 4C shows the effect of vitamin D on plasma proline. Five fasting plasma proline measurements were obtained prior to the initiation of Vitamin D supplementation (2000 IU daily), after which an additional five fasting plasma measurements were obtained over the subsequent five days. There was a trend for plasma proline to decrease following treatment (p=0.059).

FIG. 5 shows the relationship between these two measures daily chlorpromazine (CPZ) equivalents (y=axis) and neuroleptic dose (x-axis). Multiple frequencies of x,y pairs were weighted by size (dots, maximum frequency=12). The line shows the linear regression of daily CPZ equivalents on normalized daily neuroleptic dose using unweighted data (y=9.2x-19.6, adjusted r²=0.92, p<0.0001, n=63).

FIG. 6 shows a conditional effects plot of the model predicted LOHS by plasma proline (continuous) in the study group (n=45, actual proline range 87-361 μM), stratified by race (African American: solid regression line, Caucasian: dashed regression line, Hispanic: dotted regression line).

FIG. 7 shows a Mediator Model for the Association of 25-hydroxyvitamin-D insufficiency and Schizophrenia. FIG. 7A shows the direct association between insufficiency and schizophrenia with no mediator (path c). FIG. 7B shows the association mediated, in part, by the presence of hyperprolinemia (indirect paths a+b). The total effect of 25-hydroxyvitamin-D insufficiency on schizophrenia risk is defined by the sum of the indirect plus direct paths (a+b+c′).

FIG. 8 shows the relationship between 25-hydroxyvitamin-D insufficiency and hyperprolinemia. Vitamin D levels are plotted for controls and patients having schizophrenia, by hyperprolinemic status. The dashed line represents the threshold level for insufficiency. The odds of vitamin D insufficiency were significantly different across groups (LR X², 3df=10.04, p=0.018): Compared to non-hyperprolinemic controls (n=85), hyperprolinemic patients (n=17) were significantly more likely to be insufficient (OR=5.5, 95% CI 1.47-20.56, p=0.01), as were non-hyperprolinemic patients (n=47, OR=2.1, 95% CI 1.001-4.32, p=0.05), but not hyperprolinemic controls (n=5, OR=1.7, 95% CI 0.28-11.13, p=0.54). Jittered points represent individual subject data. The box indicates the interquartile range (IQR), The whiskers extend to the most extreme data point <1.5 times the IQR. Mean levels of Vitamin-D±SDs for each group are as follows: non-hyperprolinemic controls 37.39±23.04, non-hyperprolinemic patients 32.66±21.64, hyperprolinemic controls 31.4±16.59, hyperprolinemic patients 28.76±25.69.

FIG. 9 shows elevated proline in sdy−/− mice. FIG. 9A shows the adjusted means plus standard deviations plotted for wild type and sdy−/− mice for (y-axis, μM/L) plasma proline (circles), and (secondary y-axis, μM/g) cortex (triangles) and hippocampus (squares). Means were adjusted by litter, n=3 litters. *=<0.05. FIG. 9B shows that significantly lower leukocyte Prodh expression was observed in sdy (−/−) animals (p=0.02).

FIG. 10A shows the change in PRODH expression prior to- and post-treatment onset. Change in log PRODH were plotted for each subject prior to-(100%), and post-treatment onset. All of the subjects received risperidone. The mean time between blood draws was 8.3 days. PRODH was significantly upregulated between pre and post assay in a subset of patients, p=0.034 (the three upper lines). There were no significant differences in time between blood draws for those with increased PRODH (n=3), versus those with no increase (n=3, p=0.2). Pre-treatment PRODH was lower in subjects with a significant increase, compared to those with no change, but this result was not significant (p=0.3). FIG. 10B shows the relationship between change in PRODH and schizophrenia symptoms. In a linear regression model, PRODH percent change was a significant predictor of total Brief Psychiatric Rating Scale (BPRS) score approximately 8 days following treatment initiation (p=0.047), after adjusting for baseline total BPRS score. Furthermore, the linear relationship between PRODH change and adjusted BPRS score was highly significant (r=−0.92, p=0.025).

FIG. 11 shows that Prodh expression changed in neurons following rosiglitazone treatment. Primary neurons were cultured in triplicate for 24 hours, and Prodh expression measured via Taqman assay, normalized to GAPDH. Expression values were standardized to vehicle-only treatment. Clinically relevant concentrations of 1-10 μM show upregulation of PRODH. Prodh expression increased in a dose-dependent manner (p<0.05).

### DETAILED DESCRIPTION OF THE INVENTION

One embodiment of the present invention is a method for treating or ameliorating the effects of a schizophrenia-spectrum disorder. This method comprises administering to a patient in need thereof a therapeutically effective amount of a proline modulator.

As used herein, a “patient” is a mammal, preferably, a human.

As used herein, a proline “modulator” means any agent that alters the plasma proline levels. A proline modulator may be activators of PRODH or activators of peroxisomal proliferator-activated receptor gamma (PPARγ). As used herein, “activators” when used with respect to PRODH or PPARγ, means an agent that can increase the function or expression of PRODH or PPARγ.

Non-limiting examples of activators of PRODH include vitamin D, curcumin, or an analog thereof. In one aspect of this embodiment, the activator of PRODH is vitamin D or an analog thereof. In another aspect of this embodiment, the activator of PRODH is curcumin or an analog thereof.

As used herein, an “analog” of vitamin D means a chemical compound that is structurally and functionally similar to vitamin D, or (1α,25-dihydroxyvitamin D3 [1α,25(OH)2D3]). Non-limiting examples of vitamin D and analogs thereof include ergocalciferol, cholecalciferol, 22-oxacalcitriol, paricalcitol, doxercalciferol, alfacalcidol, dihydrotachysterol₂, pharmaceutically acceptable salts thereof, and combinations thereof.

As used herein, an “analog” of curcumin means a chemical compound that is structurally and functionally similar to curcumin, and curcuminoid species. Non-limiting examples of curcumin and analogs thereof include curcumin, curcuma oil, turmerone, demethoxycurcumin, bisdemethoxycurcumin, pharmaceutically acceptable salts thereof, and combinations thereof.

Non-limiting examples of activators of PPARγ include thiazolidinediones (TZD), such as troglitazone, rosiglitazone, roglitazone, ciglitazone, darglitazone, englitazone, hydroxypioglitazone, ketopioglitazone, pioglitazone, pioglitazone hydrochloride, rivoglitazone pharmaceutically acceptable salts thereof, and combinations thereof.

In the present invention, a “schizophrenia spectrum disorder” is one of a number of disorders that have some of the same symptoms as schizophrenia. Thus, “schizophrenia spectrum disorder” is intended to include schizophrenia, schizoaffective disorders, schizophreniform disorders, schizotypal and schizoid personality disorders, delusional disorders, and autism.

In one aspect of the embodiment, the method further comprises administering to the patient a therapeutically effective amount of an antipsychotic agent, a glutamatergic agent, or a combination thereof.

Non-limiting examples of antipsychotic agents of the present invention include Haloperidol, Droperidol, Chlorpromazine, Fluphenazine, Perphenazine, Prochlorperazine, Thioridazine, Trifluoperazine, Mesoridazine, Periciazine, Promazine, Triflupromazine, Levomepromazine, Promethazine, Pimozide, Cyamemazine, Chlorprothixene, Clopenthixol, Flupenthixol, Thiothixene, Zuclopenthixol, Clozapine, Olanzapine, Risperidone, Quetiapine, Ziprasidone, Amisulpride, Asenapine, Paliperidone, Iloperidone, Zotepine, Sertindole, Aripiprazole, Cannabidiol, pharmaceutically acceptable salts thereof, and combinations thereof.

Non-limiting examples of a glutamatergic agent according to the present invention include D-serine, D-cycloserine, glycine, L-proline, D-aspartate, (L- or D-glutamate, aspartate and alanine), ketamine, and phencyclidine (pcp), pharmaceutically acceptable salts thereof, and combinations thereof.

Another embodiment of the present inventions is a method of selecting a patient at risk for or suffering from schizophrenia likely to benefit from proline modulation. This method comprises:
(a) obtaining a biological sample from the patient;
(b) testing the biological sample to determine whether the patient has hyperprolinemia, wherein a patient with hyperprolinemia is a candidate for proline modulation treatment; and
(c) if the patient is determined from step (b) to have hyperprolinemia, administering to the patient an effective amount of an activator of PRODH or an activator of PPARγ. The activators of PRODH and PPARγ are as previously defined herein.

As used herein, a “biological sample” means a biological specimen, which may be a bodily fluid or a tissue. Preferred biological samples include whole blood, serum, plasma, cerebro-spinal fluid, leukocytes or leukocyte subtype cells (e.g. neutrophils, basophils, and eosinophils, lymphocytes, monocytes, macrophages), fibroblast sample, olfactory neuron sample, and tissues from the central nervous system, such as the cortex and hippocampus.

In one aspect of this embodiment, the method further comprises administering to the patient determined to have hyperprolinemia a therapeutically effective amount of an antipsychotic agent, a glutamatergic agent, or a combination thereof. Suitable antipsychotic agents and glutamatergic agents are as disclosed herein.

Yet another embodiment of the present invention is a composition for treating or ameliorating the effects of schizophrenia. This composition comprises an effective amount of a proline modulator, and a pharmaceutically acceptable carrier. Suitable proline modulators are as disclosed herein.

In one aspect of this embodiment, the proline modulator is selected from the group consisting of activators of PRODH and PPARγ. Preferably, the activator of PRODH is vitamin D, curcumin, or an analog thereof. Preferred activators of PPARγ are as disclosed herein.

In another aspect of this embodiment, the composition further comprises a therapeutically effective amount of an antipsychotic agent, a glutamatergic agent, or a combination thereof. Suitable antipsychotic agents and glutamatergic agents are as disclosed herein.

A further embodiment of the present invention is a method for identifying an agent that modulates proline levels in a patient. This method comprises:
(a) administering a candidate agent to a non-human animal having a null mutation of Dtnbp 1;
(b) carrying out an assay to determine whether the candidate agent changes the proline level or the PRODH level in the non-human animal relative to a control;
   wherein a candidate agent that causes a change in the proline level or the PRODH level of the non-human animal relative to the control is an agent that modulates proline levels in a patient.

As used herein, “Dtnbp 1” or “DTNBP 1” means the gene encoding dysbindin, or dystrobrevin-binding protein 1. A “null mutation” is an abnormal copy of a gene that completely or at least substantially lacks that gene's normal function.

As used herein, a “control” means an experiment or observation designed to minimize the effects of variables other than the single independent variable that is being tested. For example, in this embodiment, an appropriate control is an inactive substance or preparation, such as a placebo or a solvent used to dissolve the candidate agent, that is administered to a group of non-human animals having a null mutation of Dtnbp 1. Such a control serves as a comparison in order to determine the changes in the proline level that are attributable to the administration of a candidate agent. In other embodiments below, an appropriate control may be a group of individuals that serves as a comparison group when certain factors, such as e.g., proline levels, are evaluated.

As used herein, a “change” in the proline level or the PRODH level of a non-human animal means that the administration of the candidate agent resulted in a proline level or a PRODH level that is different from the original level, such as an increase or a decrease. Preferably, the candidate agent decreases the proline level in the non-human animal relative to a control. Also preferably, the candidate agent increases the PRODH level in the non-human animal relative to a control. Assays for determining proline levels and for determining PRODH levels are well-known in the art and are further disclosed below. Preferably, the assay for determining proline levels is a proline assay as defined below, more preferably a high throughput (HTP) proline assay, such as those disclosed by Grainger et al., 2004 and Le Boucher et al., 1997.

In one aspect of this embodiment, the non-human animal is a mouse; but other non-human animals may be used. Preferably, the non-human animal having a null mutation of Dtnbp1 has a sdy genotype.

In another aspect of this embodiment, the candidate agent is a biologic or a chemical. Suitable biologics and chemicals are as disclosed herein. As used herein, a “biologic” means a substance which is derived from or produced by a living organism or synthesized to mimic an in vivo-derived agent or a derivative or product thereof. A biologic may be, for example, a nucleic acid, a polypeptide, or a polysaccharide. Preferably, the biologic is a nucleic acid, a protein, or a combination thereof. As used herein, a “chemical” means a substance that has a definite chemical composition and characteristic properties and that is not a biologic. Non-limiting examples of chemicals include small organic compounds and small inorganic compounds.

Nucleic Acid

“Nucleic acid” or “oligonucleotide” or “polynucleotide” used herein mean at least two nucleotides covalently linked together.

Nucleic acids may be single stranded or double stranded, or may contain portions of both double stranded and single stranded sequences. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be synthesized as a single stranded molecule or expressed in a cell (in vitro or in vivo) using a synthetic gene. Nucleic acids may be obtained by chemical synthesis methods or by recombinant methods.

The nucleic acid may also be a RNA such as a mRNA, tRNA, short hairpin RNA (shRNA), short interfering RNA (sRNA), double-stranded RNA (dsRNA), transcriptional gene silencing RNA (ptgsRNA), Piwi-interacting RNA, pri-miRNA, pre-miRNA, micro-RNA (miRNA), or anti-miRNA, as described, e.g., in U.S. patent application Ser. Nos. 11/429,720, 11/384,049, 11/418,870, and 11/429,720 and Published International Application Nos. WO 2005/116250 and WO 2006/126040.

sRNA gene-targeting may be carried out by transient sRNA transfer into cells, achieved by such classic methods as lipid-mediated transfection (such as encapsulation in liposome, complexing with cationic lipids, cholesterol, and/or condensing polymers, electroporation, or microinjection). sRNA gene-targeting may also be carried out by administration of sRNA conjugated with antibodies or sRNA complexed with a fusion protein comprising a cell-penetrating peptide conjugated to a double-stranded (ds) RNA-binding domain (DRBD) that binds to the sRNA (see, e.g., U.S. Patent Application Publication No. 2009/0093026).

An shRNA molecule has two sequence regions that are reversely complementary to one another and can form a double strand with one another in an intramolecular manner. shRNA gene-targeting may be carried out by using a vector introduced into cells, such as viral vectors (lentiviral vectors, adenoviral vectors, or adeno-associated viral vectors for example).

The nucleic acid may also be an aptamer, an intramer, or a spiegelmer. The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), disclosed in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker (Brody, E. N. and L. Gold (2000) J. Biotechnol. 74:5-13).

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those disclosed in U.S. Pat. Nos. 5,235,033 and 5,034,506. Nucleic acids containing one or more non-naturally occurring or modified nucleotides are also included within the definition of nucleic acid. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH₂, NHR, NR₂ or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as disclosed in Krutzfeldt et al., Nature (Oct. 30, 2005), Soutschek et al., Nature 432:173-178 (2004), and U.S. Patent Application Publication No. 20050107325. Modified nucleotides and nucleic acids may also include locked nucleic acids (LNA), as disclosed in U.S. Patent Application Publication No. 20020115080. Additional modified nucleotides and nucleic acids are disclosed in U.S. Patent Application Publication No. 20050182005. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made.

Peptide, Polypeptide, Protein

The terms “peptide,” “polypeptide,” and “protein” are used interchangeably herein. In the present invention, these terms mean a linked sequence of amino acids, which may be natural, synthetic, or a modification, or combination of natural and synthetic. The term includes antibodies, antibody mimetics, domain antibodies, lipocalins, targeted proteases, and polypeptide mimetics. The term also includes vaccines containing a peptide or peptide fragment intended to raise antibodies against the peptide or peptide fragment.

“Antibody” as used herein includes an antibody of classes IgG, IgM, IgA, IgD, or IgE, or fragments or derivatives thereof, including Fab, F(ab′)2, Fd, and single chain antibodies, diabodies, bispecific antibodies, and bifunctional antibodies. The antibody may be a monoclonal antibody, polyclonal antibody, affinity purified antibody, or mixtures thereof which exhibits sufficient binding specificity to a desired epitope or a sequence derived therefrom. The antibody may also be a chimeric antibody. The antibody may be derivatized by the attachment of one or more chemical, peptide, or polypeptide moieties known in the art. The antibody may be conjugated with a chemical moiety. The antibody may be a human or humanized antibody. These and other antibodies are disclosed in U.S. Published Patent Application No. 20070065447.

Other antibody-like molecules are also within the scope of the present invention. Such antibody-like molecules include, e.g., receptor traps (such as entanercept), antibody mimetics (such as adnectins, fibronectin based “addressable” therapeutic binding molecules from, e.g., Compound Therapeutics, Inc.), domain antibodies (the smallest functional fragment of a naturally occurring single-domain antibody (such as, e.g., nanobodies; see, e.g., Cortez-Retamozo et al., Cancer Res. 2004 Apr. 15; 64(8):2853-7)).

Suitable antibody mimetics generally can be used as surrogates for the antibodies and antibody fragments described herein. Such antibody mimetics may be associated with advantageous properties (e.g., they may be water soluble, resistant to proteolysis, and/or be nonimmunogenic). For example, peptides comprising a synthetic beta-loop structure that mimics the second complementarity-determining region (CDR) of monoclonal antibodies have been proposed and generated. See, e.g., Saragovi et al., Science. Aug. 16, 1991; 253(5021):792-5. Peptide antibody mimetics also have been generated by use of peptide mapping to determine “active” antigen recognition residues, molecular modeling, and a molecular dynamics trajectory analysis, so as to design a peptide mimic containing antigen contact residues from multiple CDRs. See, e.g., Cassett et al., Biochem Biophys Res Commun. Jul. 18, 2003; 307(1):198-205. Additional discussion of related principles, methods, etc., that may be applicable in the context of this invention are provided in, e.g., Fassina, Immunomethods. October 1994; 5(2):121-9.

As used herein, “peptide” includes targeted proteases, which are capable of, e.g., substrate-targeted inhibition of post-translational modification such as disclosed in, e.g., U.S. Patent Application Publication No. 20060275823.

In the present invention, “peptide” further includes anticalins. Anticalins can be screened for agents that decrease the number of cancer stem cells. Anticalins are ligand-binding proteins that have been constructed based on a lipocalin scaffold (Weiss, G. A. and H. B. Lowman (2000) Chem. Biol. 7:R177-R184; Skerra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art, and can include conservative substitutions (e.g., substitutions that do not alter binding specificity) or substitutions that modestly, moderately, or significantly alter binding specificity.

In general, a polypeptide mimetic (“peptidomimetic”) is a molecule that mimics the biological activity of a polypeptide, but that is not peptidic in chemical nature. While, in certain embodiments, a peptidomimetic is a molecule that contains no peptide bonds (that is, amide bonds between amino acids), the term peptidomimetic may include molecules that are not completely peptidic in character, such as pseudo-peptides, semi-peptides, and peptoids. Examples of some peptidomimetics by the broader definition (e.g., where part of a polypeptide is replaced by a structure lacking peptide bonds) are described below. Whether completely or partially non-peptide in character, peptidomimetics according to this invention may provide a spatial arrangement of reactive chemical moieties that closely resembles the three-dimensional arrangement of active groups in a polypeptide. As a result of this similar active-site geometry, the peptidomimetic may exhibit biological effects that are similar to the biological activity of a polypeptide.

There are several potential advantages for using a mimetic of a given polypeptide rather than the polypeptide itself. For example, polypeptides may exhibit two undesirable attributes, i.e., poor bioavailability and short duration of action. Peptidomimetics are often small enough to be both orally active and to have a long duration of action. There are also problems associated with stability, storage and immunoreactivity for polypeptides that may be reduced with peptidomimetics.

Polypeptides having a desired biological activity can be used in the development of peptidomimetics with similar biological activities. Techniques of developing peptidomimetics from polypeptides are known. Peptide bonds can be replaced by non-peptide bonds that allow the peptidomimetic to adopt a similar structure, and therefore biological activity, to the original polypeptide. Further modifications can also be made by replacing chemical groups of the amino acids with other chemical groups of similar structure, shape or reactivity. The development of peptidomimetics can be aided by determining the tertiary structure of the original polypeptide, either free or bound to a ligand, by NMR spectroscopy, crystallography and/or computer-aided molecular modeling. These techniques aid in the development of novel compositions of higher potency and/or greater bioavailability and/or greater stability than the original polypeptide (Dean (1994), BioEssays, 16: 683-687; Cohen and Shatzmiller (1993), J. Mol. Graph., 11: 166-173; Wiley and Rich (1993), Med. Res. Rev., 13: 327-384; Moore (1994), Trends Pharmacol. Sci., 15: 124-129; Hruby (1993), Biopolymers, 33: 1073-1082; Bugg et al. (1993), Sci. Am., 269: 92-98.

Polysaccharides

The term “polysaccharides” means polymeric carbohydrate structures, formed of repeating units (either mono- or di-saccharides) joined together by glycosidic bonds. The units of mono- or di-saccharides may be the same or different. Non-limiting examples of polysaccharides include starch, glycogen, cellulose, and chitin.

Small Organic or Inorganic Molecules

The phrase “small organic” or “small inorganic” molecule includes any chemical or other moiety, other than polysaccharides, polypeptides, and nucleic acids, that can act to affect biological processes. Small molecules can include any number of therapeutic agents presently known and used, or can be synthesized in a library of such molecules for the purpose of screening for biological function(s). Small molecules are distinguished from macromolecules by size. The small molecules of this invention usually have a molecular weight less than about 5,000 daltons (Da), preferably less than about 2,500 Da, more preferably less than 1,000 Da, most preferably less than about 500 Da.

As used herein, the term “organic compound” refers to any carbon-based compound other than biologics such as nucleic acids, polypeptides, and polysaccharides. In addition to carbon, organic compounds may contain calcium, chlorine, fluorine, copper, hydrogen, iron, potassium, nitrogen, oxygen, sulfur and other elements. An organic compound may be in an aromatic or aliphatic form. Non-limiting examples of organic compounds include acetones, alcohols, anilines, carbohydrates, mono-saccharides, di-saccharides, amino acids, nucleosides, nucleotides, lipids, retinoids, steroids, proteoglycans, ketones, aldehydes, saturated, unsaturated and polyunsaturated fats, oils and waxes, alkenes, esters, ethers, thiols, sulfides, cyclic compounds, heterocyclic compounds, imidizoles, and phenols. An organic compound as used herein also includes nitrated organic compounds and halogenated (e.g., chlorinated) organic compounds. Collections of small molecules, and small molecules identified according to the invention are characterized by techniques such as accelerator mass spectrometry (AMS; see Turteltaub et al., Curr Pharm Des 2000 6:991-1007, Bioanalytical applications of accelerator mass spectrometry for pharmaceutical research; and Enjalbal et al., Mass Spectrom Rev 2000 19:139-61, Mass spectrometry in combinatorial chemistry.)

Preferred small molecules are relatively easier and less expensively manufactured, formulated or otherwise prepared. Preferred small molecules are stable under a variety of storage conditions. Preferred small molecules may be placed in tight association with macromolecules to form molecules that are biologically active and that have improved pharmaceutical properties. Improved pharmaceutical properties include changes in circulation time, distribution, metabolism, modification, excretion, secretion, elimination, and stability that are favorable to the desired biological activity. Improved pharmaceutical properties include changes in the toxicological and efficacy characteristics of the chemical entity.

Another embodiment of the present invention is a method for identifying whether a patient is at risk for developing a DTNBP1-related psychiatric illness, or whether a patient having a schizophrenia-spectrum disorder is at risk for an increased length of hospital stay. This method comprises:
(a) obtaining a biological sample from a patient;
(b) carrying out an assay to determine whether the patient has an elevated proline level compared to a control (proline assay) or a decreased PRODH expression level relative to a control (PRODH assay),
   wherein a patient with an elevated proline level or a decreased PRODH expression level in step (b) is at risk for developing a DTNBP1-related psychiatric disease and/or is at risk for an increased length of hospital stay.

As used herein, a “DTNBP1-related psychiatric illness” means a mental disorder, including a schizophrenia-spectrum disorder such as schizophrenia, that is correlated with various alleles of DTNBP1.

As used herein, “increased length of hospital stay” means a longer duration (such as more than 1%-50% or greater) of hospitalization than average of patients having a schizophrenia-spectrum disorder in general.

Assays for determining proline levels are well-known in the art. See, e.g., Wu, 1993; Inoue et al., 1996; Le Boucher et al., 1997; and Grainger et al., 2004. In addition, commercial services for such assays are also available from vendors such as ARUP Laboratories (Salt Lake City, Utah).

Assays for determining PRODH expression levels are also well-known in the art. For example, the PRODH gene may be sequenced in order to detect null or nonsense mutations. Furthermore, PRODH expression level may also be determined by measuring the amount of PRODH gene product, such as by using antibodies to the gene product. Such antibodies are commercially available from, e.g., Novus Biologicals (Littleton, Colo.) and Epitomics Inc. (Burlingame, Calif.). Preferably, PRODH expression level is determined by testing for PRODH RNA level, such as, by quantitative PCR methods, e.g., as disclosed in Turnbridge et al., 2004 and Jacob et al., 2007 or via microarray assay, for example, targeting for analysis Affymetrix probe sets 40042_r_at and 214203_s_at..

In one aspect of this embodiment, the psychiatric disease is schizophrenia.

In another aspect of this embodiment, the biological sample is whole blood, serum, plasma, cerebro-spinal fluid, leukocytes or leukocyte subtype cells (e.g. neutrophils, basophils, and eosinophils, lymphocytes, monocytes, macrophages), fibroblast sample, or olfactory neuron sample.

In a further aspect of this embodiment, the proline level is a fasting proline level.

Antipsychotic agents and/or glutamatergic agents may be administered with proline modulators together in the same composition, simultaneously in separate compositions, or as separate compositions administered at different times, as deemed most appropriate by a physician.

In the present invention, an “effective amount” or a “therapeutically effective amount” of a compound or composition disclosed herein is an amount of such compound or composition that is sufficient to effect beneficial or desired results as described herein when administered to a patient. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of a composition according to the invention will be that amount of the composition, which is the lowest dose effective to produce the desired effect. The effective dose of a compound or composition of the present invention may be administered as two, three, four, five, six or more sub-doses, administered separately at appropriate intervals throughout the day.

A suitable, non-limiting example of a dosage of a proline modulator according to the present invention may be from about 1 ng/kg to about 5000 mg/kg. In general, however, doses employed for adult human treatment typically may be in the range of 0.0001 mg/kg/day to 0.0010 mg/kg/day, 0.0010 mg/kg/day to 0.010 mg/kg/day, 0.010 mg/kg/day to 0.10 mg/kg/day, 0.10 mg/kg/day to 1.0 mg/kg/day, 1.00 mg/kg/day to about 200 mg/kg/day, 200 mg/kg/day to about 5000 mg/kg/day. For example, the dosage may be about 1 mg/kg/day to about 100 mg/kg/day, such as, e.g., 2-10 mg/kg/day, 10-50 mg/kg/day, or 50-100 mg/kg/day. The dosage of the proline modulator also may be about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1000 mg/kg, 1100 mg/kg, 1200 mg/kg, 1300 mg/kg, 1400 mg/kg, 1500 mg/kg, 1600 mg/kg, 1700 mg/kg, 1800 mg/kg, 1900 mg/kg, 2000 mg/kg, 2100 mg/kg, 2200 mg/kg, 2300 mg/kg, 2400 mg/kg, 2500 mg/kg, 2600 mg/kg, 2700 mg/kg, 2800 mg/kg, 2900 mg/kg, 3000 mg/kg, 3500 mg/kg, 4000 mg/kg, 5000 mg/kg.

With respect to proline modulators that are vitamin D and its analogs, the dosage of the proline modulator also may be denominated in International Units (IU) per day (IU/Day) and about 100 IU/day, 200 IU/day, 300 IU/day, 400 IU/day, 500 IU/day, 600 IU/day, 700 IU/day, 800 IU/day, 900 IU/day, 1000 IU/day, 1100 IU/day, 1200 IU/day, 1300 IU/day, 1400 IU/day, 1500 IU/day, 1600 IU/day, 1700 IU/day, 1800 IU/day, 1900 IU/day, 2000 IU/day, 2100 IU/day, 2200 IU/day, 2300 IU/day, 2400 IU/day, 2500 IU/day, 2600 IU/day, 2700 IU/day, 2800 IU/day, 2900 IU/day, 3000 IU/day, 3100 IU/day, 3200 IU/day, 3300 IU/day, 3400 IU/day, 3500 IU/day, 3600 IU/day, 3700 IU/day, 3800 IU/day, 3900 IU/day, 4000 IU/day, 4500 IU/day, 5000 IU/day, 5500 IU/day, 6000 IU/day, 6500 IU/day, 7000 IU/day, 7500 IU/day, 8000 IU/day, 9000 IU/day, 10,000 IU/day, 20,000 IU/day, 30,000 IU/day, 40,000 IU/day, 50,000 IU/day, 60,000 IU/day, 70,000 IU/day, 90,000 IU/day, 100,000 IU/day, 200,000 IU/day, 300,000 IU/day, 400,000 IU/day, 500,000 IU/day, 600,000 IU/day, 700,000 IU/day, 800,000 IU/day, 900,000 IU/day, 1,000,000 IU/day, 1,100,000 IU/day, 1,200,000 IU/day, 1,300,000 IU/day, 1,400,000 IU/day, or 1,500,000 IU/day. Preferably, the dosage of the vitamin D species and analogs range between about 8,000-1,500,000 IU administered on a periodic basis of dosing per day or per week or per month.

The effective dose of the proline modulator may be administered as two, three, four, five, six or more sub-doses, administered separately at appropriate intervals throughout the day.

A composition of the present invention may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, a composition of the present invention may be administered in conjunction with other treatments. A composition of the present invention may be encapsulated or otherwise protected against gastric or other secretions, if desired.

The compositions of the invention comprise one or more active ingredients in admixture with one or more pharmaceutically-acceptable carriers and, optionally, one or more other compounds, drugs, ingredients and/or materials. Regardless of the route of administration selected, the agents/compounds of the present invention are formulated into pharmaceutically-acceptable dosage forms by conventional methods known to those of skill in the art. See, e.g., Remington, The Science and Practice of Pharmacy (21^{st} Edition, Lippincott Williams and Wilkins, Philadelphia, Pa.).

Pharmaceutically acceptable carriers are well known in the art (see, e.g., Remington, The Science and Practice of Pharmacy (21^{st} Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier used in a pharmaceutical composition of the invention must be “acceptable” in the sense of being compatible with the other ingredients of the formulation and not injurious to the patient. Carriers suitable for a selected dosage form and intended route of administration are well known in the art, and acceptable carriers for a chosen dosage form and method of administration can be determined using ordinary skill in the art.

The compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the patient. Ingredients and materials suitable for a selected dosage form and intended route of administration are well known in the art, and acceptable ingredients and materials for a chosen dosage form and method of administration may be determined using ordinary skill in the art.

Compositions of the present invention suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Compositions of the present invention for rectal or vaginal administration may be presented as a suppository, which may be prepared by mixing one or more active ingredient(s) with one or more suitable nonirritating carriers which are solid at room temperature, but liquid at body temperature and, therefore, will melt in the rectum or vaginal cavity and release the active compound. Compositions of the present invention which are suitable for vaginal administration also include pessaries, tampons, creams, gels, pastes, foams or spray formulations containing such pharmaceutically-acceptable carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable carrier. The ointments, pastes, creams and gels may contain excipients. Powders and sprays may contain excipients and propellants.

Compositions of the present invention suitable for parenteral administrations comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

The following examples are provided to further illustrate the methods of the present invention. These examples are illustrative only and are not intended to limit the scope of the invention in any way.

### EXAMPLES

### Example 1

### Subjects and Recruitment

Male and female, African American, Caucasian and Hispanic patients, aged 18-65, were recruited from inpatient wards at Bellevue Hospital Center (BHC). A significant effect of valproic acid (VPA) on plasma proline level was previously reported (Jacquet et al., 2005), and therefore schizophrenic subjects treated with VPA at the time of enrollment were excluded. Patient screening and recruitment was not dependent on their length of stay in the hospital at the time of recruitment, and thus cross-sectional data were generated. Patients received a standardized hospital diet based upon ADA Guidelines of 20% protein, 25% fat and 55% carbohydrates. Psychiatric symptoms were measured using the Brief Psychiatric Rating Scale (BPRS), the Schedule for Assessment of Positive Symptoms (SAPS), the Schedule for Assessment of Negative Symptoms (SANS), and schizophrenia diagnoses were confirmed using the Structured Clinical Interview for DSM IV Disorders (SCID).

Controls were recruited from the BHC community, with recruitment targeted to reflect the patients on age, race/ethnicity, and gender. A SCID-NP interview was conducted for all controls, who were excluded if they reported symptoms from modules A-D. All subjects completed general questionnaires, self-reporting race, and documenting diagnostic and medical history information for common diseases and prescription medication use. Capacity to give informed consent was determined in accordance with the New York University (NYU) IRB regulations. After description of the study to the subjects, written informed consent was obtained from all subjects in accordance with all institutional IRB guidelines and regulations.

### Determination of Plasma Proline Levels

For all subjects, a fasting morning blood draw was performed and heparinized blood samples sent to ARUP Laboratories (500 Chipeta Way, Salt Lake City, Utah) for quantitative plasma amino acid analysis. Proline was measured in pmoles/liter (μM).

### Statistical Analysis

Group differences were tested using the Satterthwaite t-test or ANOVA with a correction for multiple testing (assuming normality of continuous variables), and using the _{X}2 or Fisher exact test where the expected cell size was b5 (categorical variables).

Tests of normality (n=154, p<0.001) and inspection of the proline distribution suggested non-symmetry with a positive skewand heavier than normal tails. Therefore, proline levels were compared across groups using the Mann-Whitney and Kruskal-Wallis non-parametric tests, and the Spearman's rank correlation coefficient to assess relationships with continuous variables. To adjust for previously reported gender differences (Jacquet et al., 2005), Jacquet et al.'s criteria were employed to define hyperprolinemic status as a proline level two standard deviations (SDs) or more above the gender-specific mean of controls (Jacquet et al., 2005).

The effect of plasma proline on five clinical measures collected was determined using a generalized linear modeling (GLM) approach, employing a maximum-likelihood estimation to summarize the relationship between hyperprolinemia and the clinical outcomes of total BPRS, SAPS, and SANS scores, age at first hospitalization, and length of hospital stay (LOHS). To model LOHS, subjects were excluded from analysis if they were transferred to another treatment facility (n=19), as discharge due to improvement could not be considered. Distributional assumptions were tested for each dependent variable using the Anderson-Darling test.

Dependent variables were transformed where necessary (SANS total: +10 units, age at first hospitalization: log transformed), prior to tests for normality. If normality was rejected (p<0.05, see Table 1), suitable distribution were selected based upon visualization of the data distribution and the Anderson-Darling goodness-of-fit test: testing whether the assumptions fit the data for each dependent variable.

TABLE 1
Distribution Fitting for Clinical Dependent Variables

| | Test of | Dis- | | |
|---|---|---|---|---|
| | Normality | tribu- | | Goodness-of-fit |
| Variable | (A-Sq) | tion | Link | (A-Sq) |
| BPRS (n = 64) | 0.49, p = 0.225 | Gaussian | Iden- | 0.49, p = 0.225 |
| | | | tity | |
| SAPS (n = 64) | 1.04, p = 0.009 | Gamma | Log | 0.38, p > 0.25 |
| SANS (n = 64) | 2.98, p < 0.005 | Gaussian | Log | 0.66, p = 0.08 |
| Age at first | 2.08, p < 0.005 | Gaussian | Log | 0.58, p = 0.131 |
| Hospitalization | | | | |
| (n = 47) | | | | |
| LOHS (n = 45) | 1.435, p < 0.005 | Gamma | Log | 0.31, p > 0.50 |

For models that passed criteria (a relationship with hyperprolinemia when alpha <0.1), medication (CPZ equivalent daily dose), severity of illness (total BPRS, SAPS, and SANS scores), history of alcohol abuse/dependence, smoking status, prior housing status before admission, plus the demographic variables age, race, gender, current occupational status (currently working or attending school compared to those currently unemployed), and highest education level reached (excluding subjects still in education) were assessed as possible covariates. Due to the cross-sectional nature of the data collection, the variable of time from admission to proline measurement was also evaluated as a covariate in the LOHS model. To assess utility in adjusting the dependent variable, each covariate was entered into a bivariate analysis, and terms found to have p values of <0.10 carried forward to a multivariate model, where the effect of plasma proline on LOHS was examined while controlling for significant potential confounding variables (p<0.05). Final model selection and fit were determined using Akaike's information criterion and the Likelihood Ratio test ([−2 ln (likelihood for null model/likelihood for alternative model)]), which tested for the significant influence of covariates plus the main explanatory variable in two sequential models. Outliers in the data were characterized by Cook's distance values (DiN4/n), and assessment of the absolute value of DFBETAs for the intercept and each independent variable. Coefficients were retransformed back to the original units. Assumptions of independence and homoscedasticity of errors were met for all models and there were no signs of multicollinearity between predictor variables.

Bonferroni corrections of final models were employed to adjust for multiple clinical measures hypothesis testing of the secondary outcomes (n=5). Statistical analysis was performed in SAS v9.1, Stata IC v10.1, and R v2.10.1.

Sample Characteristics

64 schizophrenic patients and 90 healthy controls met the study criteria and were included in the analysis. Subject's demographic characteristics are shown in Table 2. Subjects were matched on gender, ethnicity, and age. There were no significant differences between study groups on the presence of common diseases, prescription of common medications, or on alcohol or substance abuse and/or dependence. However there was a significant difference in smoking status, as more patients reported that they were current or previous smokers (p<0.0001). Schizophrenic patients were relatively short-stay inpatients (mean length of hospital stay 42±27 days), recruited following psychiatric hospitalization to the BHC primary care facility. Clinical characteristics and medication profiles of the patients are shown in Tables 3 and 4.

TABLE 2
Demographic characteristics of schizophrenic patients
(SZ) and healthy control subjects, n = 154.

| Characteristic | SZ | | Control | | |
|---|---|---|---|---|---|
| n | n = 64 | | n = 90 | | Prob^{a} |
| Females, % (n) | 51.6 | (33) | 51.1 | (46) | .9560 |
| Ethnicity, % (n) | | | | | .9775 |
| African American | 32.8 | (21) | 34.4 | (31) | |
| Caucasian | 34.4 | (22) | 33.3 | (30) | |
| Hispanic | 32.8 | (21) | 32.2 | (29) | |
| Age (years), mean ± SD | 38.5 ± 11.3 | | 37.9 ± 12.0 | | .7187 |
| Body mass index, | 27.2 ± 5.4 | | 26.4 ± 5.0 | | .3663 |
| mean ± SD | | | | | |
| Smoking status, % (n) | | | | | <0001* |
| Current or previous | 60.93 | (39) | 24.4 | (22) | |
| Never smoked | 34.38 | (22) | 74.5 | (67) | |
| Not reported | 4.69 | (3) | 1.1 | (1) | |
| History of | | | | | .7087 |
| alcoholism, % (n) | | | | | |
| Abuse | 9.4 | (6) | 6.7 | (6) | |
| Dependence | 6.3 | (4) | 4.4 | (4) | |
| Neither | 84.4 | (54) | 88.9 | (80) | |
| History of substance | | | | | .1279 |
| abuse, % (n) | | | | | |
| Abuse | 7.8 | (5) | 3.3 | (3) | |
| Dependence | 14.1 | (9) | 6.7 | (6) | |
| Neither | 78.1 | (50) | 90.0 | (81) | |
| History of | 1.6 | (1) | 0 | (0) | .4106 |
| seizures, % (n) | | | | | |
| Asthma, % (n) | 7.9 | (5) | 9.0 | (8) | .7368 |
| IDD, % (n)^{a} | 4.8 | (3) | 0 | (0) | .0692 |
| NIDD, % (n)^{c} | 7.9 | (5) | 4.4 | (4) | .4855 |
| Common | | | | | |
| medication, % (n) | | | | | |
| Antibiotics | 3.1 | (2) | 0 | (0) | .1711 |
| Antilipidemics | 6.3 | (4) | 4.4 | (4) | .7192 |
| Antihypertensives | 9.4 | (6) | 3.3 | (3) | .1644 |
| Antivirals | 1.6 | (1) | 1.1 | (1) | .9999 |
| Steroids | 6.3 | (4) | 1.1 | (1) | .1609 |
| Season of Recruitment, | | | | | |
| n (%) | | | | | |
| Winter/Spring | 34 | (53) | 49 | (54) | |
| Summer/Fall | 30 | (47) | 41 | (46) | |
| Alcohol Abuse or | 10 | (15.6) | 10 | (11.1) | 0.41 |
| dependence | | | | | |
| Hospital Duration | 12.5 | (15) | n/a | | |
| (days)^{d}, median | | | | | |
| (IQR) | | | | | |
| Fasting plasma | 215.84 ± 63.0 | | 174.28 ± 55.97 | | <0.0001 |
| proline (μM), | | | | | |
| mean ± SD | | | | | |
| Fasting Hyperprolinemia^{e} | 17 | (26.6) | 5 | (5.6) | <0.001 |
| Fasting Vitamin | 31.63 ± 22.64 | | 37.06 ± 22.7 | | 0.043 |
| D (ng/ml)^{f}, | | | | | |
| mean ± SD | | | | | |
| Fasting Vitamin D | 44 | (68.8) | 42 | (46.7) | 0.007 |
| Insufficiency^{g} | | | | | |

*= significant values when comparing SZ patients to controls.
^{a}p-values calculated by Satterthwaite t-test, Fisher exact test, or Chi-Square.
^{b} IDD = Insulin Dependent Diabetes.
^{c}NIDD = Non-Insulin Dependent Diabetes.
^{d}Days in hospital prior to fasting blood draw.
^{e}Hyperprolinemia defined as a fasting plasma proline level ± 2 SDs from the gender-specific mean of the control group: ≧203.3 μM for females and ≧327.6 μM for males.
^{f}25-hydroxyvitamin D was measured simultaneously for all subjects from stored frozen plasma samples stabilized with dithiothreitol 0.1% (w/v) final concentration, at ARUP Laboratories, 500 Chipeta Way, SLC, UT 84108.
^{g}25-hydroxyvitamin D insufficiency defined as <30 ng/ml.

TABLE 3
Clinical characteristics of schizophrenic subjects (n = 64).

| Characteristic | Mean | SD | Min | Max |
|---|---|---|---|---|
| Age at first hospitalization^{a} | 24.6 | 7.5 | 14 | 44 |
| Length of hospital stay (days) | 42.2 | 27.4 | 8 | 135 |
| BPRS^{b} Total symptoms | 32.6 | 8.2 | 18 | 56 |
| SAPS^{c} Total symptoms | 15.1 | 10.2 | 1 | 51 |
| SANS^{d} Total symptoms | 15.3 | 10.9 | 0 | 56 |

| SZ Subtype, % (n) | % (n) | |
|---|---|---|
| Disorganized | 14.1 | (9) |
| Catatonic | 0 | (0) |
| Paranoid | 34.4 | (22) |
| Residual | 4.7 | (3) |
| Undifferentiated | 46.9 | (30) |

^{a}n = 47 for whom this characteristic could be obtained.
^{b}Brief psychiatric rating scale.
^{c}Schedule for assessment of positive symptoms.
^{d}Schedule for assessment of negative symptoms.

TABLE 4
Medication of schizophrenic subjects (n = 64).

| Neuroleptic Medications Received | % (n) | |
|---|---|---|
| Neuroleptic Type | | |
| Typical only | 15.6 | (10) |
| Atypical only | 68.7 | (44) |
| Both | 14.1 | (9) |
| None | 1.6 | (1) |

| | Subjects (n) | Mean | SD | Min | Max |
|---|---|---|---|---|---|
| Neuroleptic | | | | | |
| Poly-pharmacy | | | | | |
| and Dose | | | | | |
| Total number of NL | 64 | 1.2 | 0.5 | 0 | 3 |
| Administered | | | | | |
| Daily CPZE dose^{a,c} | 63 | 550.2 | 358.3 | 0 | 2100 |
| Normalized daily | 64 | 62.7 | 37.6 | 0 | 226.9 |
| NL dose^{b,c} | | | | | |
| Medication Dose (mg) | | | | | |
| Aripiprazole | 7 | 35.0 | 21.2 | 5 | 75 |
| Clozapine | 4 | 300.0 | 196.9 | 125 | 575 |
| Fluphenazine | 1 | 10.0 | | 10 | 10 |
| Haloperidol | 16 | 9.3 | 4.5 | 1 | 20 |
| Paliperidone | 1 | 9.0 | | 9 | 9 |
| Olanzapine | 13 | 21.5 | 11.1 | 5 | 40 |
| Perphenazine | 2 | 18.0 | 8.5 | 12 | 24 |
| Quetiapine | 8 | 520.0 | 312.9 | 60 | 1050 |
| Risperidone | 24 | 4.5 | 1.4 | 1 | 8 |
| Mood stabilizers: | | | | | |
| dose (mg) | | | | | |
| Total number of | | 0.1 | 0.3 | 0 | 1 |
| mood stabilizers | | | | | |
| administered^{d} | | | | | |
| Lithium | 5 | 1140.0 | 134.2 | 900 | 1200 |
| Lamotrigine | 2 | 100.0 | 70.7 | 50 | 150 |
| None | 57 | | | | |

NLs—neuroleptic drugs.
^{a}Chlorpromazine (CPZ) equivalent dose, n = 63 as one subject's NL had no CPZ equivalent.
^{b}Percent of the n = 64 group maximum daily dose for each NL medication. The summed percentages across all NLs taken were calculated for each individual.
^{c}CPZE and normalized daily neuroleptic dose were highly correlated, r2 = 0.92, p < 0.0001 (See section on Relationship between Neuroleptic Dose Summary Measures).
^{d}patients receiving valproic acid (VPA) at the time of recruitment were excluded from analyses. Of the VPA-untreated subjects (n = 64), four subjects had received VPA during their hospitalization, but prior to their enrollment into the study (one subject: last received VPA 5 days prior to enrollment, two subjects: 10 days prior, and one subject: 14 days prior).

### Relationship Between Neuroleptic Dose Summary Measures.

To explore the potential confounding of neuroleptic dose on plasma proline level, two measures were calculated: Daily Chlorpromazine (CPZ) equivalents and normalized daily neuroleptic dose (percent of the n=64 group maximum daily dose for each neuroleptic medication, summed across all neuroleptics taken, as follows:

$${\sum }_{i = 1}^{3} \frac{{Dose}_{i}}{max [ {Dose}_{i} ]} * 100$$

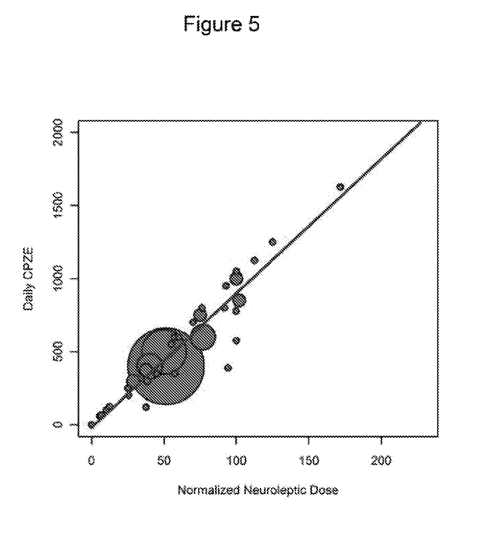

The scatter plot in FIG. 5 illustrates the relationship between these two measures (daily CPZ equivalents: y axis, normalized daily neuroleptic dose: x axis). Multiple frequencies of x,y pairs were weighted by size (dots, maximum frequency=12). The line shows the linear regression of daily CPZ equivalents on normalized daily neuroleptic dose using unweighted data (y=9.2x-19.6, adjusted r²=0.92, p<0.0001, n=63).

### Association Between Plasma Proline Level and Schizophrenia

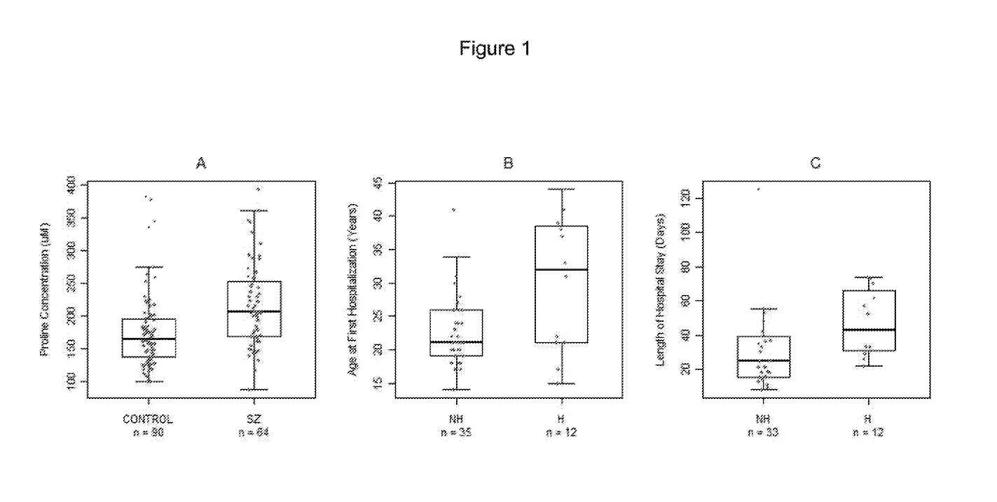

Schizophrenic patients had significantly higher fasting plasma proline levels than controls (FIG. 1A, p<0.0001). Previously, studies have reported the effects of gender (Bremer et al., 1981; Jacquet et al., 2005) and alcohol use (Walter et al., 2008) on proline level, and so the effect of these two confounds on the finding of elevated proline in schizophrenia was examined. Proline was higher in males than in females; significantly higher in controls (204.41±61.59 versus 145.46±28.9, Mann-Whitney z=5.58, p<0.0001) with a trend toward significance in the SZ group (229.26±59.17 versus 203.24±64.76, z=1.87, p=0.06). Importantly, the finding of significantly higher proline in schizophrenic patients compared to controls remained following a gender-stratified analysis (males: z=−2.35, p=0.019, females: z=−4.48, p<0.0001).

A relationship with alcohol use was only observed in controls; ten controls reporting alcohol abuse or dependence had significantly higher proline than eighty controls with no abuse or dependence (203.5±38.8 versus 170.63±56.9, Mann-Whitney z=2.55, p=0.0106). In the patient group, no significant differences were observed between alcohol use groups (z=1.507, p=0.132). These data are consistent with a recent study suggesting effects only of current alcohol on proline level (Walter et al., 2008) and are perhaps indicative of inpatient's lack of access to alcohol.

An investigation of other potential confounds was also performed. Proline levels did not differ across ethnic groups (n=154, Kruskal-Wallis _{X}2=1.955, 2df, p=0.45), or between subjects who had previously or currently smoked and those that had never smoked (n=154, Mann-Whitney z=−1.071, p=0.28). Moreover, a significant difference in proline level between schizophrenics and controls remained after stratifying analysis by smoking status (non-smokers p=0.005, current/previous smokers p=0.016). In the patient group, there was also no relationship between proline level and education (rho=−0.1056, p=0.51, n=41). As previously reported (Jacquet et al., 2005), there was no relationship between proline and age in the study sample (n=154, Spearman's rho=0.04, p=0.66).

Association of Hyperprolinemic Status with Schizophrenia

Subjects with hyperprolinemia were identified following a gender specific adjustment for proline differences (Jacquet et al., 2005). The distribution of hyperprolinemic subjects was significantly different between controls and schizophrenic subjects (n=5/90 and 17/64 respectively, 1df, OR=6.15, p=0.0003, 95% Cl 1.99-22.4). Thus, subjects with hyperprolinemia have six times greater odds of schizophrenia. This result is unlikely confounded by racial/ethnic, smoking status, or age group differences. In addition, this result remained significant following analysis where the 20 subjects who reported alcohol abuse or dependence were excluded (1df, OR=6.31, p=0.0005, 95% Cl 1.99-23.41). Of interest, there was a trend towards significance for subjects who were hyperprolinemic to be sampled in the early part of their hospitalization, compared to those who were not hyperprolinemic, who were sampled later in their hospitalization (proportion of stay=time from admission to proline measurement/total hospital stay: 0.38±0.26, n=17 versus 0.49±0.25, n=47 Mann-Whitney z=−1.7, p=0.089).

Testing the Effects of Potential Medication Confounds

As described, schizophrenic subjects were excluded from analysis if they were currently receiving the mood stabilizer VPA, due to the known influence of VPA on plasma proline (Jacquet et al., 2005). Four subjects had received VPA during their current hospitalization; although the VPA treatment had ended 5-14 days prior to enrollment into the study (see Table 4). The significant association of hyperprolinemia with schizophrenia remained after removing all four patients (1 hyperprolinemic, 3 non-hyperprolinemic patients) from analysis (n=150, 1df, OR=6.18, p=0.005). In the patient group (n=64) there was no significant difference in the proportion of hyperprolinemic patients receiving other mood stabilizer medications compared to those receiving no mood-stabilizers (p=1.0). With regards to neuroleptic use, only one patient did not receive neuroleptic medication prior to blood draw. However, as for mood-stabilizers, there was no significant difference in the proportion of hyperprolinemic subjects receiving only atypical neuroleptics compared to those receiving typicals only (n=13/44 versus n=2/10, p=0.71), and there were no proline differences in the atypical-only versus typical only groups (z=−0.56, p=0.58, n=54). There was also no relationship between proline and two independent summary measures of neuroleptic dose; daily CPZ equivalents (rho=−0.07, p=0.59, n=64), or normalized daily neuroleptic dose (see Table 4 and FIG. 5 (rho=−0.06, p=0.62, n=63)), although the measures themselves were highly correlated. In the patient group, there was no significant difference in the proportion of hyperprolinemic subjects versus non-hyperprolinemic subjects receiving the anticholinergic benztropine (n=2/17 versus n=12/47, p=0.32), and there was no relationship with proline level and benztropine dose (rho=−0.18, p=0.54, n=14). None of the control subjects reported benztropine use. Similarly, use of antidepressants (n=2/17 versus n=6/47, p=0.99), or benzodiazepines (n=3/17 versus n=8/47, p=0.99) did not differ significantly in the hyperprolinemic versus non-hyperprolinemic patient groups. In patients there was also no relationship between proline level and education (rho=−0.1056, p=0.51, n=41). These data suggest that the association between hyperprolinemia and schizophrenia does not arise from mood stabilizer and/or neuroleptic use, and is consistent with published studies (Jacquet et al., 2005).

Patient Characteristics Associated with Hyperprolinemia

Initial analysis of the data illustrated a significant relationship between hyperprolinemia and age at first hospitalization (AFH) (FIG. 1B, p=0.001). Only the variable of age passed covariate evaluation for an effect on AFH (p=0.055), but was subsequently removed from the final log-normal AFH model based upon the LRT (p>0.05). Following adjustment for gender, due to the known effects of gender on age at first onset and hospitalization (Rabinowitz et al., 2006), and a correction for multiple testing, the significant relationship remained (p=0.015). Retransformation of the hyperprolinemia coefficient predicted that hyperprolinemic patients (mean age at first hospitalization=29.9±10.2 years) were, on average, 7 years older than non-hyperprolinemic subjects (mean age=22.7±5.4) when they were first hospitalized.

A significant bivariate relationship between hyperprolinemia and LOHS (FIG. 1C, p=0.017) was also observed. For analysis of the LOHS outcome variable, subjects who were transferred or discharged to another care facility such as a state psychiatric hospital (n=19) were excluded, because these subjects may not have achieved a degree of improvement to allow for interpretation of LOHS as clinically relevant. To further model LOHS, a gamma distribution was determined a good-fit to characterize the outcome, with a log link function to specify the relationship with the explanatory variables.

Variables that passed criteria from the bivariate screen are detailed below. Specifically, to model LOHS, a gamma distribution was determined a good-fit to characterize the outcome, with a log link function to specify the relationship with the explanatory variables (see Table 1 above). In an initial gamma-log screen, it was found that the explanatory variables of time (p<0.001), BPRS total score (p<0.022), prior housing status (private residence versus group home, p=0.275; private residence versus homeless, p=0.03), alcohol use (p=0.023), and race (Hispanic versus Caucasian, p=0.011; Hispanic versus African American, p=0.002) passed criteria of p<0.1, as shown in Table 5. These variables were thus carried through to a multivariate model, which also included the independent binary variable of hyperprolinemia. The covariates of prior housing status and alcohol use did not remain significant (p>0.05) and were excluded from the final main effects model (Table 6 below).

TABLE 5
Bivariate Analysis for Between-Subject Effects (n = 45) on LOHS^{a}

| Variable | df | b | se | z | p |
|---|---|---|---|---|---|
| Time^{b} | 1 | 0.027 | 0.0106 | 2.55 | 0.011 |
| Race^{c} | 2 | | | | |
| Hispanic V Caucasian | | 0.7596 | 0.2918 | 2.60 | 0.009 |
| Hispanic V African | | 0.689 | 0.2488 | 2.77 | 0.006 |
| American | | | | | |
| BPRS Total Score | 1 | 0.03 | 0.0129 | 2.33 | 0.020 |
| Prior Hospitalization status^{d} | 2 | | | | |
| Private Residence V Group | | −0.4997 | 0.4113 | −1.21 | 0.224 |
| Shelter | | | | | |
| Private Residence V | | 0.7185 | 0.2501 | 2.87 | 0.004 |
| Homeless | | | | | |
| Alcohol Dependence/abuse^{e} | 1 | −0.559 | 0.246 | −2.27 | 0.023 |

^{a}Bivariate model with a gamma distribution and log link
^{b}Time from admission to blood draw and proline measurement (days)
^{c}Nominal variable with three levels
^{d}Nominal variable with three levels
^{e}Binary variable, yes/no

Hyperprolinemic status was found to have a significant effect on the outcome of LOHS, when adjusted for the time to blood draw, BPRS score, and race (Table 6), and further adjustment for multiple testing (p=0.005). Retransformation of the coefficients predicted that patients with hyperprolinemia stayed in the hospital on average an additional 13 days longer than non-hyperprolinemic patients, keeping the variables of time, BPRS, and race constant.

TABLE 6
Multivariate modeling of length of hospital stay (LOHS).
Final model**^{a} Log likelihood (LL): −168.19

| Variable | b | se | z | p^{b} | LR χ² | p^{c} |
|---|---|---|---|---|---|---|
| Constant | 1.67 | 0.251 | 6.66 | <0.001 | | |
| Time^{d} | 0.029 | 0.005 | 5.92 | <0.001 | 40.38 | <0.001 |
| (mean = 14.91 days) | | | | | | |
| Race | | | | | | |
| Hispanic (n = 15) v | 0.322 | 0.132 | 2.45 | 0.014 | | |
| Caucasian (n = 16) | | | | | | |
| Hispanic (n = 15) v | 0.586 | 0.132 | 4.43 | <0.001 | | |
| African American | | | | | | |
| (n = 14) | | | | | | |
| BPRS total score | 0.028 | 0.007 | 3.87 | <0.001 | | |
| (mean = 32.3) | | | | | | |
| Hyperprolinemia | 0.408 | 0.120 | 3.38 | 0.001 | 10.67 | 0.0011 |
| (Yes, n = 12 v No, | | | | | | |
| n = 33) | | | | | | |

**n = 45, df = 5, X² = 51.05, p < 0.0001.
^{a}Gamma distribution with log link.
^{b}Probability > N|z|.
^{c}Probability of likelihood ratio (LR) statistic (p > Nχ²).
^{d}Time from admission to proline measurement.

Because of the small sample size, model interactions with the main effects were not statistically evaluated.

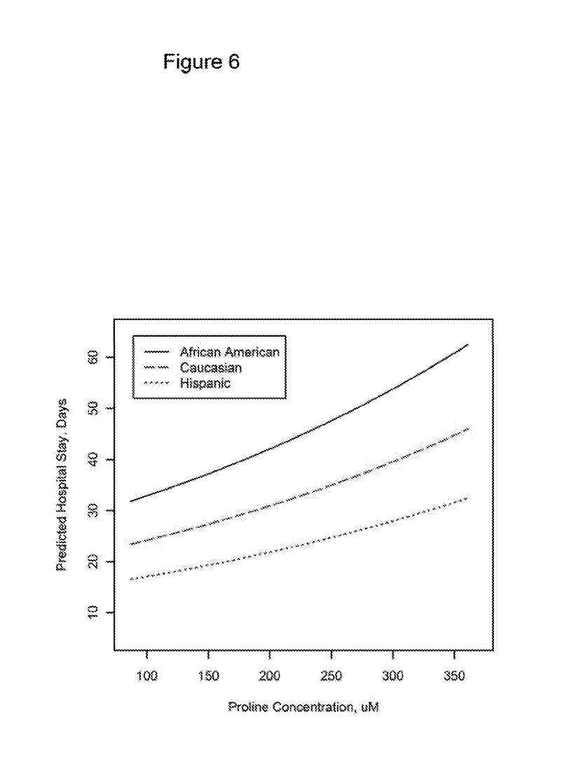

Hyperprolinemic status had a significant effect on the outcome of LOHS (Table 6, p=0.001), when adjusted for the time from admission to blood draw, total BPRS score, and race/ethnicity. Because of the small sample size, model interactions with the main effects were not statistically evaluated. However, a potential interaction with race and hyperprolinemia was observed, as there appeared to be a greater predicted effect of elevated proline on hospital stay in African American patients, when compared to Hispanic patients (p=0.043), but not in Caucasian compared to Hispanic patients (p=0.94). To optimally visualize the effect between race and proline elevation on LOHS, a conditional effects plot of the model predicted LOHS was generated (FIG. 6)).

There was no significant difference in the proportion of subjects with hyperprolinemia across schizophrenia subtypes (disorganized n=3/9, catatonic 0/0, paranoid 5/22, residual 2/3, undifferentiated 7/30, p=0.38), and proline levels did not differ across subtype (Kruskal-Wallis _{X}2=0.75, 3 df, p=0.86). In the patient group, there was no significant bivariate relationship between hyperprolinemia and measures of symptom severity: BPRS total (1df, p=0.48, n=64), SAPS total (1df, p=0.40, n=64), or SANS total score (1df, p=0.40, n=64).

Vitamin D Levels

Vitamin-D modulates gene expression. While screening the NIH GEO database, in vitro PRODH upregulation in response to 1alpha,25-dihydroxy-vitamin-D3 was observed (Accession GSE5145; probe set 214203_s_at). From this finding, it was hypothesized that schizophrenia risk may be mediated by proline elevation due to vitamin D deficits. Fasting plasma 25-hydroxyvitamin-D in 64 schizophrenia patients and 90 matched controls were therefore measured. These individuals were previously assayed for fasting plasma proline (Table 1 and Clelland et al., 2011). The relationship between Vitamin-D and hyperprolinemia was investigated (Table 1).

Vitamin-D levels were significantly lower in patients (Table 1, z=2.023, p=0.043), and 25-hydroxyvitamin-D insufficiency (<30 ng/ml), was significantly associated with schizophrenia (OR 2.51, 95% Cl: 1.3-4.9). Ethnicity and season of recruitment were independent predictors of insufficiency, although neither confounded the relationship between insufficiency and diagnosis in a multivariate logistic model (p>0.05 for all covariates, likelihood ratio test p=0.913, see below for model description). Age, gender, vitamin supplementation, smoking status, alcohol use, and for patients, time in hospital and CPZ-equivalents, were not predictors of vitamin-D insufficiency.

25-hydroxyvitamin-D levels were negatively correlated with fasting proline (n=154, rho=−0.21, p=0.01). Furthermore, subjects with 25-hydroxyvitamin-D insufficiency had three times greater odds of hyperprolinemia than those with optimal levels (p=0.035, 95% Cl: 1.08-8.91). Hyperprolinemic status thus fulfilled criteria for mediating the association between insufficiency and schizophrenia, and formal testing of the indirect versus direct effects confirmed this hypothesis. Controlling for hyperprolinemia decreased the strength of the direct association between 25-hydroxyvitamin-D insufficiency and schizophrenia (OR: 2.17, 95% Cl: 1.08-4.35), with nearly one third of this relationship (31.2%) mediated by the presence of hyperprolinemia.

Bivariate Analysis for Independent Predictors of 25-Hydroxyvitamin-D Insufficiency.

In an initial screen, it was found that the explanatory variables of ethnicity (African American versus Caucasian, β=−1.21, p=0.004; African American versus Hispanic, β=−0.49 p=0.24), and season (winter/spring versus summer/fall, β=0.57, p=0.083), were predictors of 25-hydroxyvitamin-D insufficiency, at p<0.1, and thus passed criteria to be included in the multivariate modeling. The following variables were determined to have no relationship with the outcome of vitamin-D insufficiency: Age (p=0.99), gender (p=0.49), alcohol use (p=0.13), vitamin D supplementation (≦400 IU/day, p=0.73), BMI (p=0.27), smoking status (p=0.25), and for the patients, duration of their hospital stay prior to the fasting blood draw (p=0.38) and chlorpromazine (CPZ) equivalents (p=0.434).

Although multiple studies have documented the stability of Vitamin D after decades of storage, two variables related to the age of the plasma samples were investigated to confirm these results. In this study, 25-hydroxyvitamin-D was measured simultaneously for all subjects at the study end, and therefore the number of days in storage at −70° C. ranged from 746-1516 days for the fasting plasma samples. In addition, following recruitment of the first 56 subjects (n=23 patients and n=33 controls), the form of the reducing agent dithiothreitol (DTT), added to the plasma samples was changed from powder to solution, 0.1% w/v final concentration. The variable of number of days in storage from blood draw to 25-hydroxyvitamin-D measurement did not predict vitamin-D insufficiency (β=0.0008, p=0.317), however, DTT form predicted insufficiency (β=0.71, p=0.036), and so was taken forward into the multivariate logistic model.

Multivariate Modeling of Schizophrenia Risk and Mediation.

A multivariate logistic model was used to determine the effect of 25-hydroxyvitamin-D insufficiency on the outcome of diagnostic group (Table 7), with adjustment for covariates that passed the bivariate screen (a relationship with Vitamin D insufficiency, p<0.1). Model goodness-of-fit was determined using the Likelihood Ratio test of sequential models: 2 versus 1, 3 versus 2, and 4 versus 3.

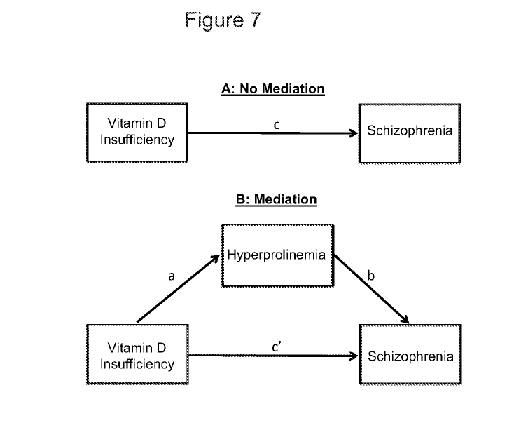

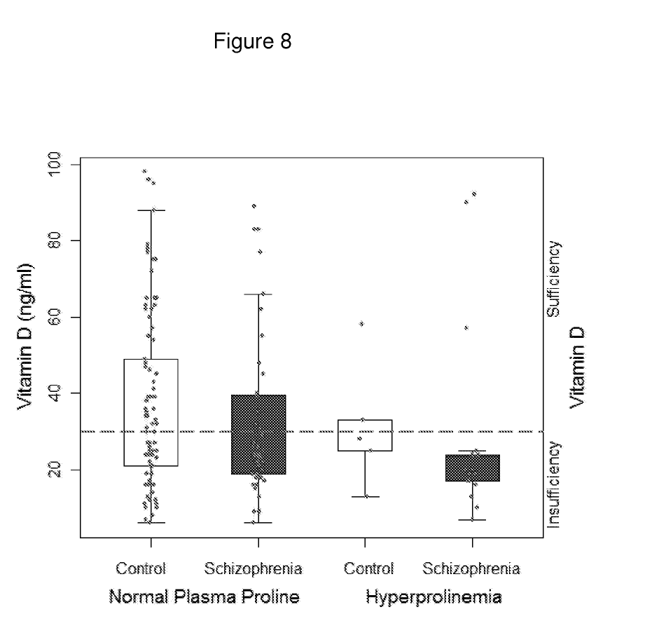

The hypothesis that hyperprolinemia mediated the relationship between 25-hydroxyvitamin-D insufficiency and schizophrenia was also investigated (FIG. 7). The standardized coefficients for paths c, a, b, and c′ were calculated from the relevant models, with bias-corrected confidence intervals for the coefficients calculated from 500 bootstrap replications (Table 8). The proportion of the total effect (a+b+c′) mediated by the presence of hyperprolinemia (a+b/a+b+c′) was significant at 31.2%

TABLE 7
Modeling Multivariate Predictors of Schizophrenia (n = 154).

| Covariate | OR | se | z | p^{a} | LR χ² | p^{b} |
|---|---|---|---|---|---|---|
| Model 1 | | | | | | |
| (χ² = 8.5, df = 5, | | | | | | |
| p = 0.13) | | | | | | |
| Vitamin D Insufficiency | 2.79 | 1.01 | 2.84 | 0.005 | | |
| Race | | | | | | |
| African American v | 1.47 | 0.62 | 0.91 | 0.365 | | |
| Caucasian | | | | | | |
| African American v | 1.21 | 0.51 | 0.47 | 0.640 | | |
| Hispanic | | | | | | |
| Season | 0.998 | 0.40 | -0.00 | 0.996 | | |
| DTT form | 0.86 | 0.36 | -0.35 | 0.723 | | |
| Model 2 | | | | | 0.98 | 0.913 |
| (χ² = 7.52, df = 1, | | | | | | |
| p = 0.0061) | | | | | | |
| Vitamin D Insufficiency | 2.51 | 0.86 | 2.69 | 0.007 | | |
| Model 3 | | | | | 10.92 | 0.0009 |
| (χ² = 18.44, df = 2, | | | | | | |
| p = 0.0001) | | | | | | |
| Vitamin D Insufficiency | 2.17 | 0.77 | 2.18 | 0.030 | | |
| Hyperprolinemia | 5.33 | 2.92 | 3.05 | 0.002 | | |
| Model 4 | | | | | 0.12 | 0.730 |
| (χ² = 18.56, df = 3, | | | | | | |
| p = 0.0003) | | | | | | |
| Vitamin D Insufficiency | 2.08 | 0.78 | 1.96 | 0.050 | | |
| Hyperprolinemia | 4.06 | 3.88 | 1.47 | 0.143 | | |
| Interaction | 1.49 | 1.75 | 0.34 | 0.732 | | |
| (insufficiency * | | | | | | |
| hyperprolinemia) | | | | | | |

^{a}probability > |z|
^{b}probability of likelihood ratio (LR) statistic (p > χ²)

TABLE 8
Testing for Mediation by Hyperprolinemia^{a}(n = 154)

| | Standardized | Bootstrap | | |
|---|---|---|---|---|
| Pathway | coefficient^{b} | se | 95% Confidence Interval^{c} | |
| Indirect (a + b) | 0.0888 | 0.0521 | 0.0087 | 0.2015 |
| Direct (c′) | 0.1961 | 0.0916 | 0.0058 | 0.3749 |
| Total (a + b + c′) | 0.2848 | 0.0956 | 0.0769 | 0.4530 |
| No mediation (c) | 0.2455 | | | |

^{a}Test for Binary mediation performed in Stata v11.0.
^{b}Rescaling based upon the variable SDs, to allow indirect effects to be computed as the product of coefficients.
^{c}Bias-corrected CI. Significant if 95% CI does not contain zero.

### CONCLUSIONS

Schizophrenic patients had significantly elevated fasting plasma proline levels, compared to matched control subjects. The confounding effects of alcohol and gender on plasma proline (1981; Jacquet et al., 2005; Walter et al., 2008), were evaluated: alcohol abuse and dependence analysis confirmed that alcohol use did not drive the finding of elevated proline in patients, and gender-stratified analysis demonstrated a significant plasma proline elevation in schizophrenia, in both males and females. This is consistent with a report by Tomiya et al., who measured serum proline elevation in both male and female schizophrenic patients when compared to controls (Tomiya et al., 2007), although the small sample size they employed likely contributed to the insignificant finding in males.

A categorical analysis of proline was also performed. Using criteria to define gender-adjusted mild to moderate hyperprolinemia (Jacquet et al., 2005), a highly significant association with schizophrenia was demonstrated, with 26.6% of the patients defined as hyperprolinemic compared to 5.6% of controls. Potential medication-based confounds on this association were investigated. VPA-treated patients were excluded from the study and non-VPA mood stabilizer use did not have a significant effect on proline level. While the effect of neuroleptics on proline was difficult to truly assess because all but one schizophrenic patient was receiving neuroleptics, there was no evidence to suggest the proportion of hyperprolinemic subjects differed in the atypical versus typical neuroleptic use groups. There was also no relationship between proline level and two independent measures of neuroleptic dose. In summary, elevated proline and mild hyperprolinemia were significantly associated with schizophrenia in this inpatient sample, and this finding is unlikely confounded by gender, alcohol use, or patient medication.

Interestingly, an association of hyperprolinemia with schizoaffective disorder but not with schizophrenia was previously reported (Jacquet et al., 2005). Jacquet et al's., predominately paranoid schizophrenic sample had subtypes different to those reported here (p<0.001), although differences in proline level across subtypes were not detected. Additionally, Jacquet et al. sampled a Caucasian population, whereas African American, Caucasian and Hispanic subjects were recruited for this study. Although no significant differences between ethnic/racial groups on proline level were found, the possibility that the different subject groups, coupled with recruitment from different treatment settings (Jacquet et al., 2005), may account for the discrepant findings cannot be ruled out. One potential limitation of the study design was that data on the socioeconomic status of all study subjects was not collected and analyzed. However, in the patient group there was no relationship between proline level and the highest level of education reached. Moreover, proline levels were measured following an overnight fast, and therefore potential influences of socioeconomic status on, for example, diet, may be reduced, lessening the impact on the primary finding of an association between schizophrenia and hyperprolinemia.

Considering sources of the proline elevation, PRODH gene variants are a potential candidate, as variants have been identified in schizophrenia. For example PRODH variants were found in 36% of a schizophrenic patient sample (Jacquet et al., 2002), of which approximately 40% would be predicted to have low enzyme activity and elevated proline (Bender et al., 2005). There is also a strong association between schizophrenia and 22q11 DS and/or microdeletions of 22q11 encompassing the PRODH locus (Karayiorgou et al., 2010), and it has been suggested that 22q11 DS may be underdiagnosed (McDonald-McGinn et al., 2005). However, whilst the study subjects were not genotyped for PRODH variants, based upon the frequency of subjects with hyperprolinemia (26.6%), abnormal proline homeostasis may also be implicated, rather than higher than expected prevalence of 22q11 DS or functional PRODH nucleotide variants (Guilmatre et al., 2010).

This is one of the first studies to explore the association between hyperprolinemia and clinical characteristics in a schizophrenic patient sample. While hyperprolinemia was not associated with total, positive, or negative symptoms, schizophrenic patients with hyperprolinemia are significantly older at their first psychiatric hospitalization (29.9 years) when compared to non-hyperprolinemic patients (22.7 years), after adjusting for gender. Although not an exact measure of onset, previous studies have shown a strong relationship between age at first hospitalization and age of onset in both genders ((Rabinowitz et al., 2006) and references therein), and thus the finding suggests a later age of onset in subjects with elevated proline.

Interestingly, the largest study of VCFS patients reported a significantly later onset of schizophrenia in the 22q11 DS patients (mean age 26 years) compared to a control group of unrelated schizophrenic patients (mean age 19 years)(Murphy et al., 1999). That study, along with the data disclosed herein, may thus point to etiological differences between patients with and without hyperprolinemia: clinically elevated peripheral proline is reflected by elevation in the CNS (Dingman and Sporn, 1959; Efron, 1965; Baxter et al., 1985; Gogos et al., 1999; Jacquet et al., 2003; Shanti et al., 2004), and it was hypothesized that the elevated plasma proline in schizophrenia also reflects elevated CNS levels in these subjects. Speculatively, it may be that chronically elevated CNS proline increases risk for development of schizophrenia, but that long-term exposure is necessary for this effect to manifest.

It was also found that the presence of mild to moderate hyperprolinemia in schizophrenic patients predicts a significantly longer hospital stay. LOHS is a useful measure of time to clinical benefit and discharge (Centorrino et al., 2004; Wassef et al., 2005), and the finding of hospitalizations that were on average two weeks longer for hyperprolinemic subjects, which represents nearly 50% longer hospitalization periods, highlights a subset of patients with substantial increases in life disruption and inconvenience, and has important clinical and economic ramifications. For example, LOHS has been employed to compare first and second generation antipsychotics, to characterize use and outcome of antipsychotics formulations, to investigate effectiveness of delayed versus immediate release drug formulations and the benefits of polypharmacy. Although a caveat to this data interpretation arises due to the cross-sectional nature of the study measures, the significant finding remained after adjustment for the time from admission to proline measurement. Intriguingly, the data also showed a trend towards significance for hyperprolinemic subjects to be sampled earlier in their hospital stay, when compared to non-hyperprolinemic subjects. A longitudinal study investigating proline level over the course of an individual patient's hospitalization, that also explores the relationship with clinical improvement (as measured by the change in a clinical severity scale, such as the BPRS) between admission and discharge, would be an optimal and warranted approach to further explore the findings.

Proline has several properties that are similar to classical excitatory amino acid neurotransmitters, such as its release at the synapse after K⁺-induced depolarization, its synthesis within synaptosomes and its uptake into synaptosomes by a high-affinity Na-dependent transport system (Nickolson, 1982; Yoneda and Roberts, 1982; Nadler, 1987; Nadler et al., 1992). In addition, the PROT high affinity proline transporter is differentially expressed in a subpopulation of excitatory nerve terminals and proline can modulate glutamatergic neurotransmission, further supporting a CNS neurotransmission-related role for proline (Fremeau et al., 1992; Shafqat et al., 1995; Velaz-Faircloth et al., 1995; Cohen and Nadler, 1997a, 1997b; Renick et al., 1999; Phang et al., 2001). Based upon the significant findings disclosed herein of elevated proline in patients with schizophrenia, of later age at first hospitalization in hyperprolinemic subjects, and if confirmed, the finding that hyperprolinemia is associated with delayed patient hospital discharge following improvement, it was hypothesized that elevated proline is a risk factor for schizophrenia and may represent an intermediate phenotype of a distinct etiological subtype of the disorder, providing insight into the etiology of schizophrenia and potentially a target for new therapeutic strategies. Further study of hyperprolinemia in schizophrenia, and also schizoaffective disorder (Jacquet et al., 2005), is warranted to elucidate whether proline elevation and a theorized dysregulation of CNS neurotransmission propagates the disease or symptom onset, or is simply a marker of psychiatric illness.

This study provides a mechanism by which 25-hydroxyvitamin D insufficiency confers risk of schizophrenia; via proline elevation and the concomitant dysregulation of neurotransmission. Insufficiency of Vitamin-D has been implicated in schizophrenia susceptibility, although the mechanism by which this deficit confers risk is unknown. We performed a formal test of causal mediation, and showed that nearly one third of the association between Vitamin-D insufficiency and schizophrenia, can be explained by the presence of hyperprolinemia. The results of this work provides a mechanism by which 25-hydroxyvitamin D insufficiency confers risk of schizophrenia; via reduced PRODH expression, proline elevation, and the concomitant dysregulation of neurotransmission. This study also implicates hyperprolinemia in the disturbance of dopamine signaling observed in DVD neonates.

### Example 2

To explore the genetic basis of hyperprolinemia in SZ, DNA and leukocyte RNA will be collected from patient and control subjects. These genetic material will be used to: a) sequence the PRODH gene and test for association of variants with elevated proline at admission, and b) quantitate peripheral PRODH transcripts and test for association of RNA level and/or alternatively spliced mRNAs with elevated proline. The exploratory hypothesis that normalization of hyperprolinemia during hospitalization may be due, in part, to regulation of PRODH transcription will also be tested. Identification of PRODH variants and/or expression dysregulation associated with hyperprolinemia will provide evidence of a genetic basis for, and support an etiological role of, hyperprolinemia in SZ.

Several PRODH mutations have previously been shown to cause decreased PDX activity, and if the genetic analysis finds association of PRODH gene variants with hyperprolinemia in SZ, this finding will indicate a mechanism for the observed hyperprolinemia, cementing a role for PRODH and variants in the gene and/or loss of the region encompassing PRODH on chromosome 22, and, significantly, providing new insight into the etiology of SZ.

When considering sources of the proline elevation, PRODH gene variants are a potential candidate, as variants have been identified in SZ and associated with hyperprolinemia, there is a strong association between SZ and 22q11 DS and/or microdeletions of 22q11 encompassing the PRODH locus 26, and it has been suggested that 22q11 DS may be under-diagnosed. This study will be one of the largest screens of PRODH, and also has the potential to identify non-coding variants that may alter mRNA levels. This genotype-phenotype interaction analysis in over 500 subjects greatly expands similar studies, such as a 2010 study of 19 HP1 patients.

Furthermore, testing for association of PRODH variants (and expression dysregulation) with hyperprolinemia rather than SZ, should allow for more definitive conclusions to be drawn, as compared to PRODH SZ association studies due to extensive reduction in heterogeneity of the outcome measure.

PRODH Variant Screening.

The PRODH gene spans over 23.7 kb of chromosome 22q11, and comprises 15 exons. The longest transcript (at 2.4 kb), encodes isoform 1, consisting of 600 amino acids. Isoform 2, consisting of 492 amino acids, is encoded by a transcript missing an internal exon at the 5′ end. A PRODH pseudogene, which lies telomeric to the functional copy, has >95% sequence identify. To target the functional copy, a large internal deletion in the pseudogene will be used when designing the Fluidigm Arrays for target enrichment (see below), and an initial long-range PCR strategy for selective amplification of PRODH will be incorporated.

The Fluidigm Access Array™ System.

One of the largest problems with high-throughput, next generation sequencing (via, for example, the Roche 454 FLX system that will be employed for these studies), is the need to capture the target sequence from every individual. Previously this could involve hundreds of individual PCRs from genomic DNA to generate the “sequencing library”, and was very much a rate-limiting, and expensive step. For this project, the innovative Access system will be used: arrays target 48 individual samples per batch, and within each sample in the array, contain bar-coded primer sets designed for specific amplification of the PRODH gene (and designed against pseudogene sequences). Due to the multiplex nature of the system, and the amount of primer sets per well/array, the entire gene (including 1 kb upstream and downstream, and 100 bp of intron/exon boundary for each of the exons), will be targeted in one well. Thus analysis of about 500 subjects will require only 11 arrays. A simple amplicons tag turns the bar-coded products into a 454FLX sequencing library.

DNA and RNA Extraction, and PRODH Screening.

DNA will be extracted from blood using standard procedures (Qiagen). RNA will be extracted from leukocytes, that have been processed immediately postblood draw (via initial RBC lysis and centrifugation, that stabilizes leukocytes for long-term storage). These methods are routinely employed by the inventor, and e.g. leukocytes have been found to be extremely stable, with high quality RNA extracted >2 yrs following initial storage. RNA quality will be determined using an Agilent Bioanalyzer, only RNA with RINs>7.0 will be processed further.

Preliminary Analyses, for DNA Assay, Missing Genotype Data, HWE.

Quality assurance will be performed to assess, e.g., missing genotypes and Hardy-Weinberg equilibrium (HWE). Prior to any association analysis, we will perform a set of quality control checks, including determining genotyping failure rate, minimum allele frequency, and HWE, using HAPLOVIEW and PLINK. We will exclude variants with missing genotyping in >20% of the samples, and will drop variants that deviate from HWE at p-value <0.0001 in controls. When a subject has been identified carrying multiple PRODH variants, we will determine whether they occur in cis or trans, via individual PCR amplification, subcloning and sequence analysis.

Assay of Plasma Proline.

Sample preparation and proline assay will be performed as described. Assay of 25 control or mild-moderate hyperprolinemic subjects showed minimum variation within subjects.

Detection of PRODH Transcripts.

PRODH is expressed in the periphery. TaqMan® assays will be employed (both standard plus custom designed assays for splice variant detection). Assays are based on a reporter dye (e.g. 6-FAM) linked to the 5′ end of a sequence-specific probe, designed to hybridize to an expression target between forward and reverse priming sequences. A non-fluorescent quencher, linked to the 3′ probe, suppresses the dye fluorescence. During amplification, the probe is cleaved by the 5′ exonuclease activity of Taq, and the reported dye released and quantified. Amplification conditions include 900 mM each primer and 250 nM probe, plus enzyme (ABI). Input cDNA will be normalized with an endogenous control assay (18S, using a VIC-labeled probe for duel assay). For each subject, 100 ng RNA will be employed for cDNA synthesis (1 ng of cDNA will be initially employed for the expression assays), using random oligonucleotide primers for first strand synthesis (according to the recommended manufacturer's instructions). In a multiplex dye-format, TaqMan® assays (PRODH plus housekeeper to normalize input RNA) will be performed in triplicate.

The findings from this work will provide the basis for further studies, such as 1) an investigation of hyperprolinemia in prodomal patients, such as those who participate in the Center of Prevention & Evaluation (COPE) at Columbia, an outpatient research program for teenagers and young adults at risk for psychiatric illness, 2) an investigation to determine the genetic contribution of PRODH to hyperprolinemia in SaD and BPD patients, 3) studies to explore the hypothesis that elevated peripheral proline in humans is similarly elevated in the CNS (as shown in animal model studies), via measurement of proline in CSF, and 4) it has been considered that there is relatively little transport of proline from the periphery into the CNS. However, there is an active transporter that transports proline across the blood-brain barrier, into the blood. Speculatively, a diet deficient in proline, and concomitant lower blood levels may stimulate proline efflux via the ATA2 transporter, decreasing CNS proline concentration, and thus future work would ultimately include trialing a proline deficient diet in hyperprolinemic patients with SZ.

### Example 3

Based upon the significant findings of hyperprolinemia in a large subset of patients with SZ (26%), of later age at first hospitalization in hyperprolinemic subjects, and the finding that hyperprolinemia is associated with delayed patient clinical improvement and discharge, it was hypothesized that elevated proline is a risk factor for SZ and may represent an intermediate phenotype of a distinct etiological subtype, providing a novel target for therapeutic strategies. If treatments that decrease proline level show efficacy in reducing SZ symptoms, this approach has the potential to provide targeted treatment to nearly one third of all SZ patients.

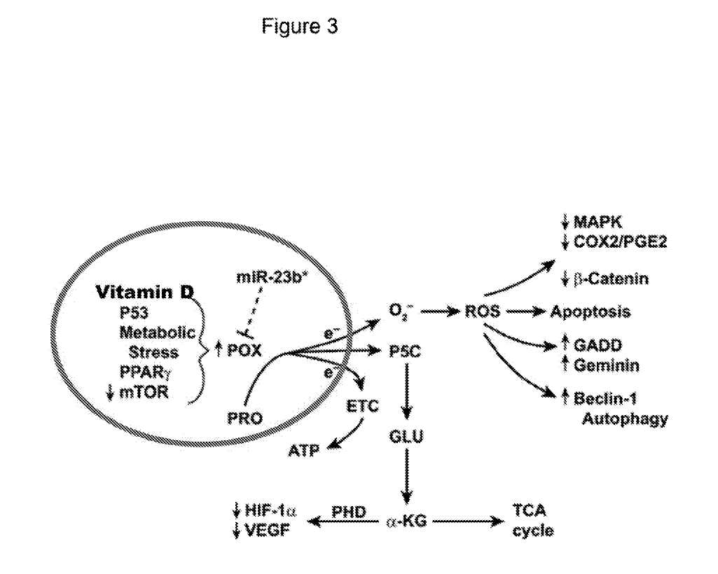

The preferred biotherapeutic agent would target the hyperprolinemia observed in >25% of SZ patients, via increasing expression of the PRODH gene and PDX activity (FIG. 3). For example, the anti-diabetic thiazolidinedione (TZDs) drugs have been found to increase PRODH gene expression via activation of the transcription factor peroxisomal proliferator-activated receptor gamma (PPARγ). However, the side-effects associated with currently available TZDs, likely due to the induction of many genes involved in lipid and glucose metabolism, suggest that new screening approaches such as that employed by Waki et al., to for example, identify targets that selectively regulate expression of PPARγ, may be required to further develop this class of biotherapeutic agent.

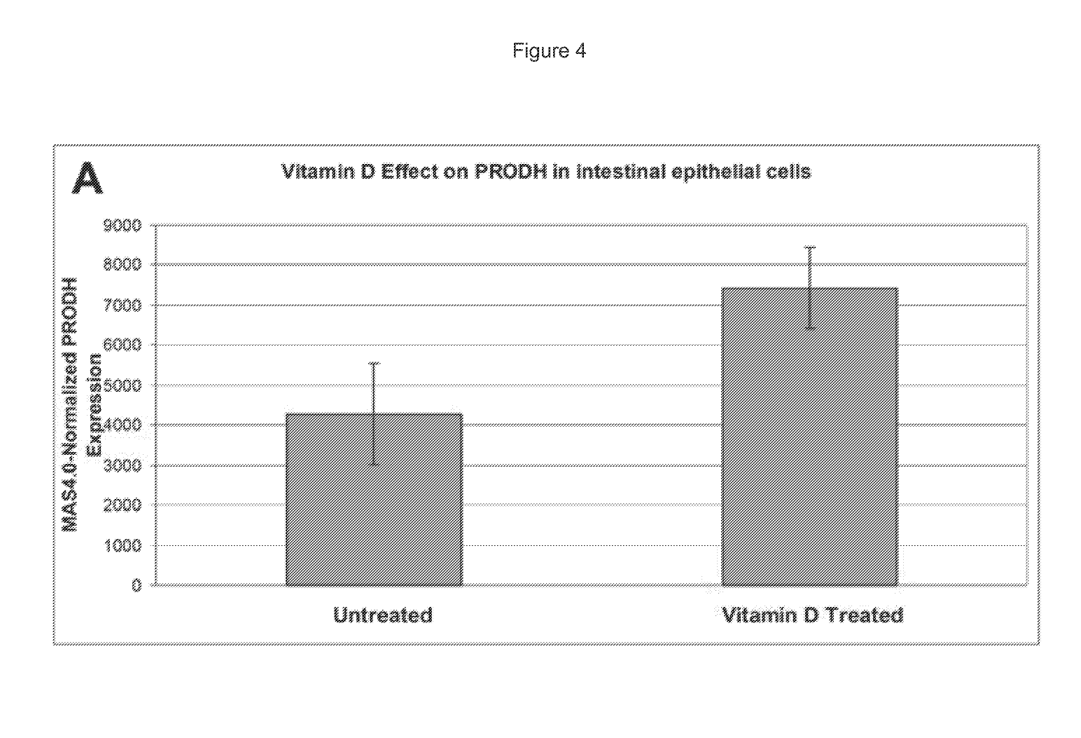

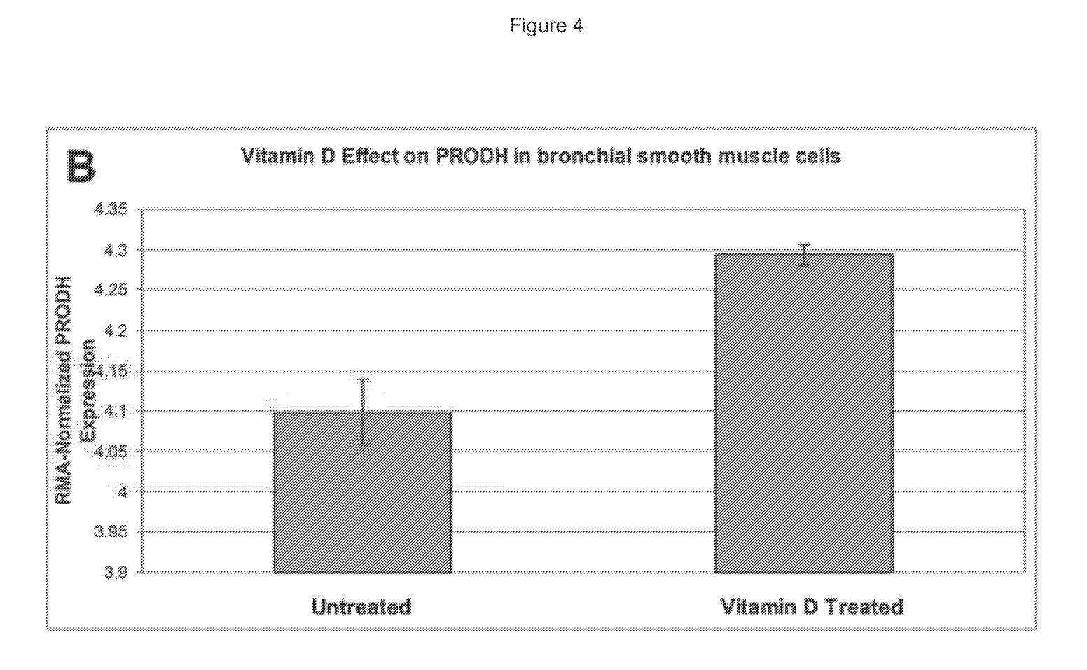

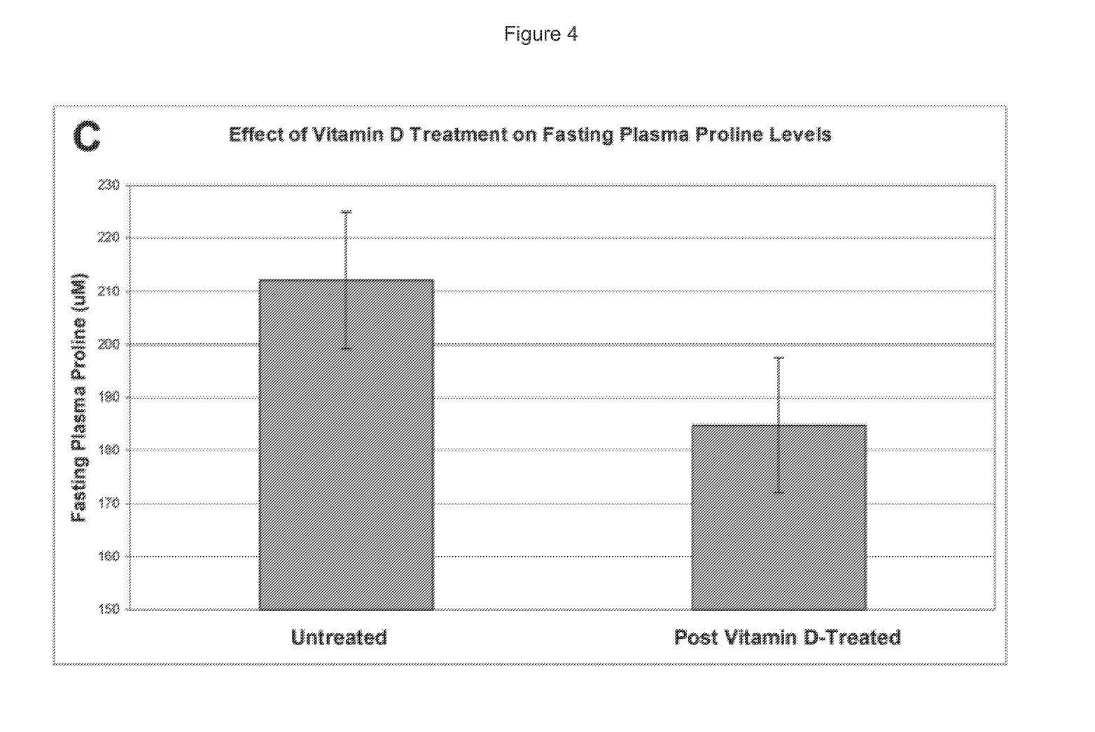

As illustrated by FIG. 4, the biologically active form of vitamin D (1α,25-dihydroxyvitamin D3 [1α,25(OH)2D3]) is also a potent regulator of PRODH expression. Specifically, analysis of microarray data from the GEO expression database shows that vitamin D significantly upregulates in vitro PRODH expression in a) intestinal epithelial cells, and b) bronchial smooth muscle cells. Moreover, in vivo (FIG. 4C), proline levels are decreased by treatment with oral vitamin D.

Vitamin D has multiple properties that lend to it's suitability as a biotherapeutic candidate for targeting hyperprolinemia: it is well tolerated with minimal side-effects, and supplementation may have preventative benefit leading researchers to suggest maternal, or early childhood supplementation therapy for those at risk for SZ. Moreover, based upon data from a developmental vitamin D deficiency model, supplementation may target both cognitive and positive symptoms, and this data is supported by a large cohort study showing that women with high dietary vitamin D consumption had a 37% lower risk of psychosis-like symptoms compared to women with low consumption. However, vitamin D toxicity in the form of hypercalcemia has been reported, and those with impaired kidney function may be at increased risk. Thus, and as suggested by Kalueff et al., the development of novel low-calcemic analogs and synthetic vitamin D analog drugs with tissue-specific uptake, may be beneficial in the development of a vitamin D based therapy for SZ.

Patient Selection for POM Study.

26% of SZ patients were found to be hyperprolinemic in the initial study (defined as having a fasting plasma proline level two standard deviations (SDs) or more above the gender-specific mean of controls). The preliminary study found no evidence that antipsychotic medications affect plasma proline levels, and we propose to perform the POM using vitamin D or a related biotherapeutic molecule, as an adjunctive treatment with SZ patients receiving stable medications over a six week study period. The candidate biotherapeutic will target proline elevation, via upregulation of PRODH expression and thus PDX activation. Subjects will be selected for POM studies based upon their positive hyperprolinemic status measured via a fasting blood draw.

Measures of Biological and Clinical Endpoints, Biological Endpoint.

It is hypothesized that treatment with the biotherapeutic candidate, such as vitamin D, will result in an upregulation of PRODH gene expression and PDX enzyme activity, and a concomitant decrease in proline level for hyperprolinemic SZ patients. To measure the biological endpoint, a surrogate tissue will be used. PRODH is expressed in the periphery, and the hyperprolinemia measured in the murine mutant Prodh E453X CNS is also reflected in peripheral tissue. Therefore, the two biological endpoints, for which there is a wealth of experience in accurately measuring, are: 1) an increase in peripheral blood leukocyte PRODH expression level after treatment, and 2) a decrease in plasma proline level, and normalization of hyperprolinemia status.

Measures of Biological and Clinical Endpoints, Clinical Endpoint.

The primary clinical efficacy outcome measures include both clinical and neurocognitive measures: the Positive and Negative Symptom Scale (PANSS), a rating scale widely used in assessment of medication effects in SZ (and references therein) and the composite score of the MATRICS Consensus Cognitive Battery. It is hypothesized that the change in the PANSS score and the MATRICS scale will be significantly higher in the treatment group (fasting hyperprolinemic patients treated with Vitamin D or related biotherapeutic, compared to the control group (hyperprolinemic placebo-treated patients). Finally, in the initial study, it was observed that hyperprolinemic subjects had significantly longer hospitalization periods (a mean of 14 days longer), and there was a trend towards significance for hyperprolinemic patients to be sampled earlier in their hospital stay, when compared to non-hyperprolinemic patients. Thus, length of hospital stay (LOHS) will also be employed as a clinical endpoint.

Clinical Differentiation from Other Therapies.

By stratifying patients based upon hyperprolinemic status, the biotherapeutic treatment will be targeted, thus separating the approach from current therapies that do not consider the heterogeneous etiology of the illness. Additionally, there is data to support a cognitive benefit of vitamin D or related treatments, which is significant because there are currently no medications approved for the treatment of cognitive symptoms in SZ.

1) Pre-clinical Objectives: Provide Biological Validity to Support an Etiological Role of SZ-Associated Hyperprolinemia.

Methods will involve screening for mutations in the PRODH gene and tests for association of PRODH gene variants with elevated proline, plus quantitation of peripheral PRODH transcripts and tests for association of RNA level and/or alternatively spliced mRNAs with elevated proline. Reagents and assays (DNA sequence analysis and Taqman assays), are available to support this objective.

2) Pre-clinical Objectives: Elucidate the Mechanisms Underlying Vitamin D Regulation of Proline.

Cell culture assays would be developed to explore in vitro the molecular mechanism of PRODH upregulation, moving to, for example, in vivo organotypic slice culture assays to explore CNS tissue specificity.

Assays to identify the optimal drug dose would be performed, using the biological endpoints described above. Animal models, such as the maternal vitamin D deficient rat model, would also be employed to directly test the hypothesis of PRODH upregulation after vitamin D supplementation.

3) Pre-clinical Objectives: Identify Novel Biotherapeutics that Target Proline Elevation.

Phage display libraries will be screened to identify peptides that displayed high affinity and selectivity for the vitamin D receptor (VDR), or in an analogous manner, for PPARγ. These strategies would be initiated if, for example, it was determined from studies performed that only high vitamin D doses would achieve normalization of hyperprolinemic status (such as greater than 4000 IU daily).

Clinical Objectives.

Clinical objectives include 1) to evaluate an anticipated clinical response to adjunct vitamin D or related treatment including negative symptoms and cognitive deficits; 2) to evaluate the safety of treatment for SZ patients; and 3) to evaluate the relationship of changes in peripheral PRODH expression and plasma proline level with efficacy outcomes. These objectives will be accomplished by conducting a double blind, six week placebo controlled trial, in which hyperprolinemic subjects with SZ will be randomized to vitamin D (or related treatment) or placebo as a treatment, adjunct to their antipsychotic medication. Baseline and end of trial assessments will be performed, and the hypothesis that the change in the PANSS total score and the MATRICS consensus cognitive scale will be significantly greater in the treatment group compared to the control group will be tested. Data on all adverse events will be collect, and the null hypothesis that the proportion of subjects in the vitamin D or related treatment group experiencing an event will not be different from the control group will be tested. PRODH expression in PBLs and plasma proline levels will also be measured, and their relationship with clinical symptoms and cognitive deficits at baseline and at study end will be examined.

It is expected that the results of this experiment will deliver a vitamin D or related treatment that normalizes the hyperprolinemia observed in close to one third of SZ patients, resulting in a clinically relevant response as shown by symptom reduction. The successful completion of this work will lead the way to a future large scale efficacy study, and it is expected that a novel treatment for SZ will be developed. Such treatment will be well tolerated with limited side effects, improved outcomes, and decreased time to clinical improvement for patients with this severe and debilitating illness, and as such has significant and important public health implications.

### Example 4

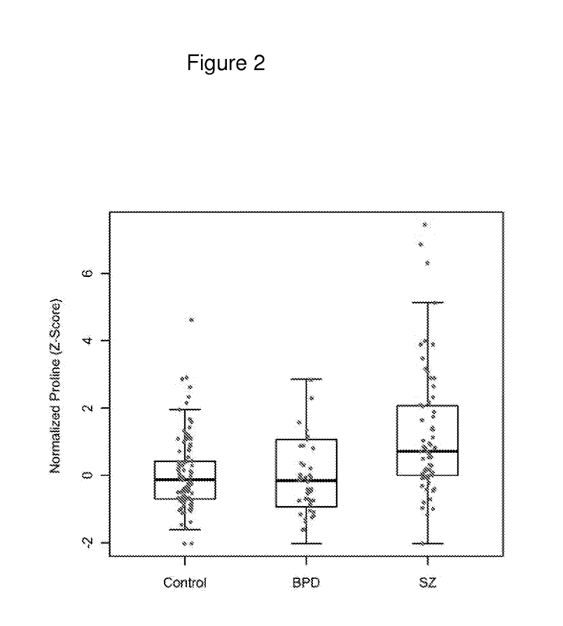

Data showing specificity of hyperprolinemia to schizophrenia-spectrum disorders were obtained (FIG. 2). Furthermore, PRODH expression was shown to rise after the onset of treatment in first-episode patients that were hypothesized to be hyperprolinemic at baseline (See below for more detail). These findings again confirm the results set forth in Examples 1-3 above.

### Dtnbp1 and Hyperprolinemia.

Additionally, it was also hypothesized that the phenotype of the dysbindin-1 (Dtnbp1) null “schizophrenia mouse model” arises (at least in part) due to hyperprolinemia that itself results from loss of Prodh regulation. This hypothesis was tested, and it was found that the Dtnbp1 null animal (the sdy genotype mice) indeed exhibited both peripheral and CNS (cortex and hippocampal) hyperprolinemia compared to wild type littermates. Reduced Prodh expression in peripheral blood leukocytes in sdy mice compared to wild type littermates was also measured. Thus, the Dtnbp1 model may be used for research in the development of treatments designed to address proline abnormalities. Furthermore, certain DTNBP1 gene variants may lead to hyperprolinemia, and the DTNBP1 gene in humans may be targeted to upregulate PRODH to treat hyperprolinemia. Additionally, a proline assay (or a Prodh expression assay) may be used to diagnose or predict risk of DTNBP1-related psychiatric illness such as schizophrenia.

Dtnbp1-Deficient Animal Procedures.

Homozygous (sdy−/−) mice (n=11) and wild type littermates (n=8) on the DB/2J background were investigated at 2 months of age. Cortical and hippocampal tissue were dissected on ice under RNAse free conditions. One dissected half cortex and hippocampus was sent to the Analytical Psychopharmacology Laboratory at The Nathan S. Kline Institute for Psychiatric Research (NKI) for proline measurement by HPLC. The other halves were employed for RNA extraction using a standard Trizol method. mRNA levels of the Prodh gene and housekeeping gene Gapdh, were assessed via quantitative RT-PCR using a SYBR-green dye as follows. 25 ng of RNA was employed for first strand cDNA synthesis and PCR performed by monitoring in real time the increase in fluorescence of the SYBR Green dye, using a Bio-Rad iQ5 machine. Additionally, 500 μl to 1 ml of whole blood was collected from each animal into EDTA-containing tubes. Blood was processed for plasma (via centrifugation) and leukocyte separation, and for proline measurement or Prodh expression assay as described.

Results. Dtnbp1-Deficient_Mice Exhibit CNS and Peripheral Hyperprolinemia:

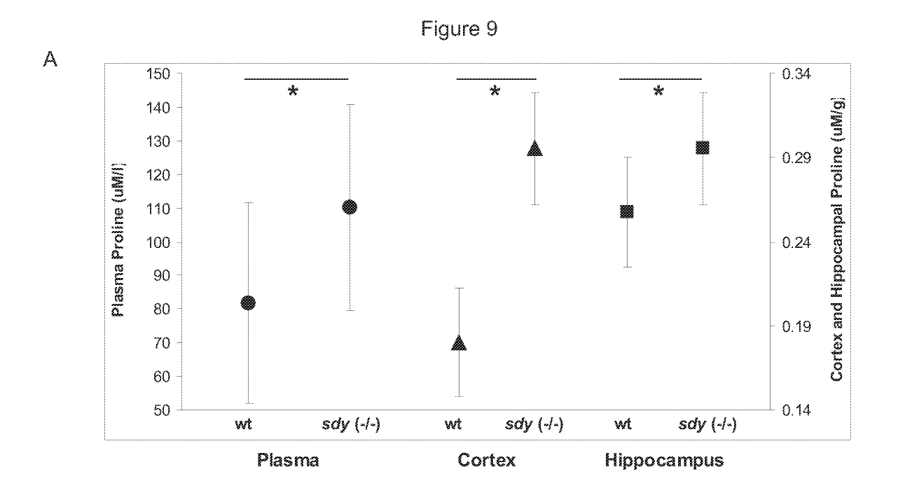

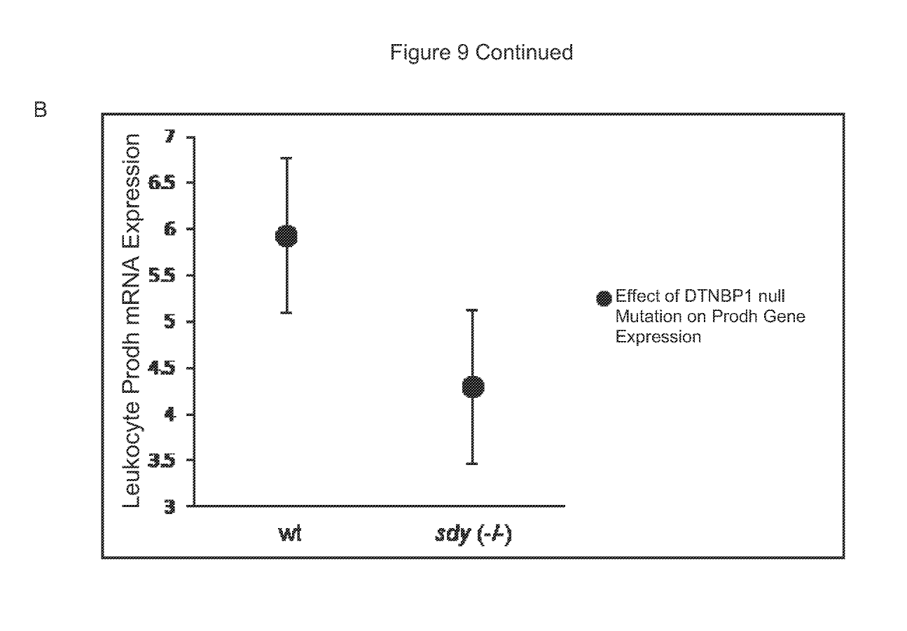

As shown in FIG. 9, proline was significantly elevated in the periphery (p=0.035), cortex (p=0.012) and hippocampus (p=0.049) in sdy−/− mice as compared their wild type littermates. In addition, peripheral Prodh transcript levels (normalized to Gapdh) were significantly lower in sdy−/− mice (FIG. 9b, p=0.02), and levels were correlated to plasma proline (n=14, r=−0.5, p=0.06).

Conclusion.

Sdy−/− mice exhibit both peripheral and CNS hyperprolinemia. Significantly lower levels of Prodh gene expression also supports our hypothesis of proline elevation through loss of Dtnbp1 regulation of the p53 transcriptional pathway.

### PRODH Expression in First-Episode Schizophrenia.

Exploratory analysis of PRODH expression levels were also performed in first-episode, never-medicated SZ patients (n=6). Male, first-episode, never-medicated SZ patients were recruited at the Bellevue Hospital CPEP. A 15 ml blood sample was collected at admission to the CPEP, and peripheral PRODH expression was measured using Affymetrix U133v2.0 arrays (PRODH is one of the 54,000 transcripts). A post-treatment measurement of PRODH expression was also obtained (mean time between blood draws=8.3 days).

Results.

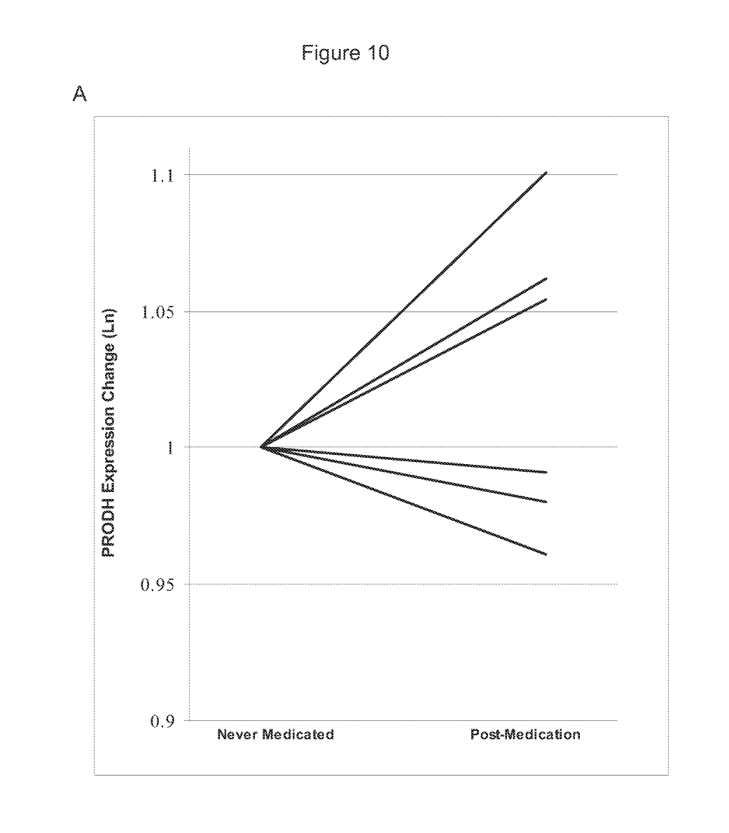

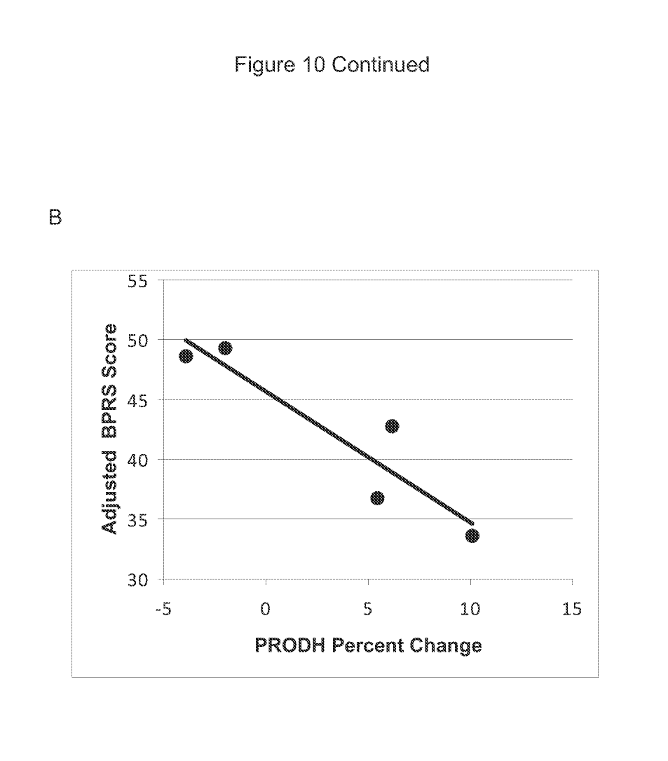

Although there was no significant change between pre and post expression levels (mean pre level=6.10±0.41 expression units; post level=6.24±0.35 expression units, n=6, p=0.3), inspection of the data suggested that for a subset of subjects (n=3, FIG. 10A, the three upper lines), PRODH expression was significantly increased after the onset of treatment (mean percent change of 7.3%, p=0.04). A slight, non-significant decrease of 2.3% was observed in the no-change group (n=3, FIG. 10A, the three bottom lines, p=0.15).

The relationship between PRODH expression and symptoms was also examined in these first-episode patients, both upon admission, and then following the initiation of treatment. The Brief Psychiatric Rating Scale (BPRS) was used as the primary measure of symptoms. Of interest, percent change in PRODH expression (post-pre/pre level×100) was a significant predictor of post-treatment total BPRS score in a linear regression model, after adjusting for admission BPRS, β=−1.09, p=0.047 (n=5, because for one subject pre-treatment BPRS measures were not available). Thus, for example, for every 10% increase in PRODH expression, total post BPRS decreases by 10.9 points, after adjusting for baseline. The relationship between percent change in PRODH and post-treatment BPRS is shown in FIG. 10B.

Conclusions.

A subset of first-episode patients had significant post-treatment upregulation of PRODH. This subset also had lower baseline PRODH at admission, thus it is intriguing to hypothesize that these subjects were baseline hyperprolinemic. We also found a significant relationship between increased PRODH and symptom improvement following treatment with risperidone alone. Thus, these data illustrates that PRODH expression may rise in some first-episode patients after treatment (all subjects received risperidone), and analysis of CNS PRODH expression in mice exposed to antipsychotics e.g. clozapine, indicates PRODH upregulation (GEO²⁴ accession GDS2531, probeset 141769_at).

### Upregulation of PRODH by RZG.

Thiazolidinedione treatment of primary neurons upregulates PRODH in a dose-response manner, and thus supports the use of this class of medication to treat hyperprolinemia in schizophrenia patients, via PRODH upregulation.

First, additional replication will be performed in in vitro neurons, using varying doses of different thiazolidinedione (TDZs) drugs to identify the most favorable response, as determined by expression of the Prodh gene. Additionally, the Dtnbp1 murine model of hyperprolinemia that exhibits a psychiatric phenotype will be used, and the homozygous sdy mutant animals will be treated with TDZ drugs (using a dose based upon the in vitro work above). Response will be initially measured using a biomaker (reduction of plasma proline and/or loss of hyperprolinemic status and/or upregulation of Prodh). The behavioral and cognitive deficits reported in these mice are expected to be alleviated/reduced compared to untreated homozygous mutation mice.

TDZs, also known as glitazones, are a class of medications, some of which have FDA approval to treat type 2 diabetes. TDZs act by activating peroxisome proliferator-activated receptors (PPARs), specifically PPAR gamma. When activated, the PPARgamma receptor regulates transcription of multiple genes, including Prodh (see, for example, Phang et al., 2010). The class of TDZ medications include rosiglitazone (RZG), roglitazone, ciglitazone, darglitazone, englitazone, hydroxypioglitazone, ketopioglitazone, pioglitazone, pioglitazone hydrochloride, and rivoglitazone.

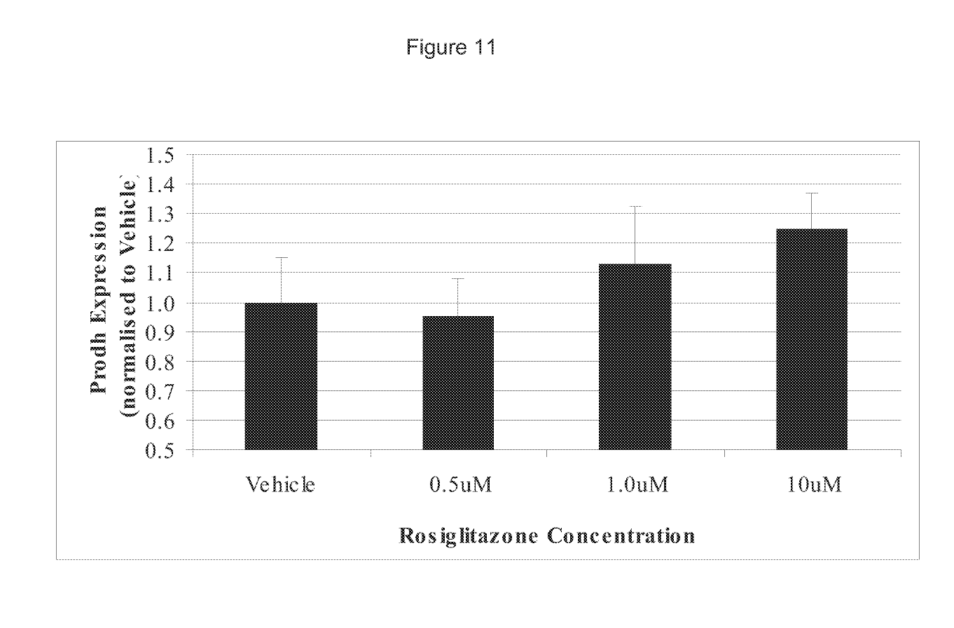

From in vitro studies, RZG was found to upregulate PRODH levels by about 400% (GDS2705). This finding was tested ex vitro utilizing primary neurons obtained from E18 mouse embryos treated with RZG. Dissociated mouse neurons were plated on culture dishes coated with poly-L-lysine (500K cells/well), and maintained for 5 days in a 5% CO₂ incubator at 37° C., prior to treatment (RZG at 0.5 μM, 1.0 μM and 10 μM). 24 hours following treatment RNA was extracted, and Prodh gene expression measured via Taqman assay, normalized to Gapdh (as described above). Expression values were standardized to vehicle-only treatment. Testing the hypothesis that there is a linear increase of PRODH across the log treatment groups, we observed that clinically relevant concentrations of 1-10 μM induced a significant upregulation of Prodh (p=0.036) (FIG. 11).

As set forth above, the main indication for TDZs are metabolic disorders, such as type 2 diabetes. TDZs have also been trialed in schizophrenia. Specifically, pioglitazone was studied for its effect on treating glucose and lipid abnormalities. Smith et al. have reported preliminary data from a placebo-controlled intervention study of pioglitazone in SZ and schizoaffective (SaD) patients receiving olanzapine or clozapine, and who had elevated fasting glucose and triglycerides. They observed significantly decreased PANSS psychopathology depression factor scores in the pioglitazone-treated subjects (mean PANSS depression score 11 pre-treatment and 8 post-treatment, p=0.01), and small but significant decreases (at 3 months vs. baseline) in Total PANSS Scores and Negative and General PANSS Scores (Robert Smith, Personal communication, and see trial information: “Pioglitazone as a Treatment for Lipid and Glucose Abnormalities In Patients With Schizophrenia”).

Regulation of Proline Via Turmeric and Curcumin Species.

Curcuminoids can upregulate PRODH expression (Ramachandran-C et al., 2005), potentially via regulation of P53 (Lee, 2009), and these findings indicate potential utility for proline modulation treatment.

By stratifying patients based upon hyperprolinemic status, treatment will be targeted, and thus this approach will be distinct from current therapies that do not consider the heterogeneous etiology of the illness. Furthermore, a model for pre-clinical testing has been identified.

A potential biomarker for SZ diagnosis or to determine at risk status may be based upon elevated proline level. Additionally, the data set forth in Example 1 above showed that hyperprolinemic status had a significant effect on a patient's length of hospital stay (p=0.005). Hospitalizations were on average two weeks longer for hyperprolinemic subjects, which represents nearly 50% longer hospitalization periods. Thus, proline levels could be employed as a biomarker to identify patients with increased hospital stays, which has potential economic ramifications.

### DOCUMENTS

Afenjar, A., Moutard, M. L., Doummar, D., Guet, A., Rabier, D., Vermersch, A. I., et al., 2007. Early neurological phenotype in 4 children with biallelic PRODH mutations. Brain Dev. 29 (9), 547-552.
Baxter, C. F., Baldwin, R. A., Davis, J. L., Flood, J. F., 1985. High proline levels in the brains of mice as related to specific learning deficits. Pharmacol. Biochem. Behay. 22 (6), 1053-1059.
Bender, H. U., Almashanu, S., Steel, G., Hu, C. A., Lin, W. W., Willis, A., et al., 2005. Functional consequences of PRODH missense mutations. Am. J. Hum. Genet. 76 (3), 409-420.
Braff, D., Stone, C., Callaway, E., Geyer, M., Glick, I., Bali, L., et al., 1978. Prestimulus effects on human startle reflex in normals and schizophrenics. Psychophysiology 15 (4), 339-343.
Bremer, H. J., Duran, M., Kamerling, J. P., Przyrembel, H., Wadman, S. K., et al., 1981. Disturbances of Amino Acid Metabolism: Clinical Chemistry and Diagnosis. Urban & Schwarzenberg, Inc., Baltimore-Munich.
Centorrino, F., Goren, J. L., Hennen, J., Salvatore, P., Kelleher, J. P., Baldessarini, R. J., 2004. Multiple versus single antipsychotic agents for hospitalized psychiatric patients: case-control study of risks versus benefits. Am. J. Psychiatry 161 (4), 700-706.
Clelland C L, Read L L, Baraldi A N, et al. Evidence for association of hyperprolinemia with schizophrenia and a measure of clinical outcome. Schizophr Res 2011; 131(1-3):139-45.
Cohen, S. M., Nadler, J. V., 1997a. Proline-induced potentiation of glutamate transmission. Brain Res. 761 (2), 271-282.
Cohen, S. M., Nadler, J. V., 1997b. Proline-induced inhibition of glutamate release in hippocampal area C A1. Brain Res. 769 (2), 333-339.
Davis, J. L., Pico, R. M., Flood, J. F., 1987. Differences in learning between hyperprolinemic mice and their congenic controls. Behay. Neural Biol. 48 (1), 128-137.
Dingman, W., Sporn, M. B., 1959. The penetration of proline and proline derivatives into brain. J. Neurochem. 4 (2), 148-153.
Efron, M. L., 1965. Familial hyperprolinemia. Report of a second case, associated with congenital renal malformations, hereditary hematuria and mild mental retardation, with demonstration of an enzyme defect. N. Engl. J. Med. 272, 1243-1254.
Flynn, M. P., Martin, M. C., Moore, P. T., Stafford, J. A., Fleming, G. A., Phang, J. M., 1989. Type I I hyperprolinaemia in a pedigree of Irish travellers (nomads). Arch. Dis. Child. 64 (12), 1699-1707.
Fremeau Jr., R. T., Caron, M. G., Blakely, R. D., 1992. Molecular cloning and expression of a high affinity L-proline transporter expressed in putative glutamatergic pathways of rat brain. Neuron 8 (5), 915-926.
Gogos, J. A., Santha, M., Takacs, Z., Beck, K. D., Luine, V., Lucas, L. R., et al., 1999. The gene encoding proline dehydrogenase modulates sensorimotor gating in mice. Nat. Genet. 21 (4), 434-439.
Goodman, B. K., Rutberg, J., Lin, W. W., Pulver, A. E., Thomas, G. H., 2000. Hyperprolinaemia in patients with deletion (22)(q11.2) syndrome. J. Inherit. Metab. Dis. 23 (8), 847-848.
Grainger et al., A microtitre format assay for proline in human serum or plasma. Clin Chim Acta. 2004; 343(1-2):113-8.
Guilmatre, A., Legallic, S., Steel, G., Willis, A., Di Rosa, G., Goldenberg, A., et al., 2010. Type I hyperprolinemia: genotype/phenotype correlations. Hum. Mutat. 31 (8), 961-965.
Humble M B, Gustafsson S, Bejerot S. Low serum levels of 25-hydroxyvitamin D (25-OHD) among psychiatric out-patients in Sweden: relations with season, age, ethnic origin and psychiatric diagnosis. J Steroid Biochem Mol Biol 2010; 121(1-2):467-70.
Inoue H, Date Y, Kohashi K, Yoshitomi H, Tsuruta Y. Determination of total hydroxyproline and proline in human serum and urine by HPLC with fluorescence detection. Biol Pharm Bull. 1996; 19(2):163-6.
Jacob et al., Alterations in Expression of Glutamatergic Transporters and Receptors in Sporadic Alzheimer's Disease. Journal of Alzheimer's Disease. 11 (1), 97-116 (2007).
Jacquet, H., Raux, G., Thibaut, F., Hecketsweiler, B., Houy, E., Demilly, C., et al., 2002. PRODH mutations and hyperprolinemia in a subset of schizophrenic patients. Hum. Mol. Genet. 11 (19), 2243-2249.
Jacquet, H., Berthelot, J., Bonnemains, C., Simard, G., Saugier-Veber, P., Raux, G., et al., 2003. The severe form of type I hyperprolinaemia results from homozygous inactivation of the PRODH gene. J. Med. Genet. 40 (1), e7.
Jacquet, H., Demily, C., Houy, E., Hecketsweiler, B., Bou, J., Raux, G., et al., 2005. Hyperprolinemia is a risk factor for schizoaffective disorder. Mol. Psychiatry 10 (5), 479-485.
Karayiorgou, M., Gogos, J. A., 2004. The molecular genetics of the 22q11-associated schizophrenia. Brain Res. Mol. Brain Res. 132 (2), 95-104.
Karayiorgou, M., Simon, T. J., Gogos, J. A., 2010. 22q11.2 microdeletions: linking DNA structural variation to brain dysfunction and schizophrenia. Nat. Rev. Neurosci. 11 (6), 402-416.
Kempf, L., Nicodemus, K. K., Kolachana, B., Vakkalanka, R., Verchinski, B. A., Egan, M. F., et al., 2008. Functional polymorphisms in PRODH are associated with risk and protection for schizophrenia and fronto-striatal structure and function. PLoS Genet. 4 (11), e1000252.
Le Boucher J, Charret C, Coudray-Lucas C, Giboudeau J, Cynober L. Amino acid determination in biological fluids by automated ion-exchange chromatography: performance of Hitachi L-8500A. Clin Chem. 1997; 43(8 Pt 1):1421-8.
Yongkyu Lee. Activation of apoptotic protein in U937 cells by a component of turmeric oil. BMB reports 2009; 42(2) pp. 96-100
McGrath J. Is it time to trial vitamin D supplements for the prevention of schizophrenia? Acta Psychiatr Scand 2010; 121(5):321-4.
McDonald-McGinn, D. M., et al., 2005. The 22q11.2 deletion in African-American patients: an underdiagnosed population? Am. J. Med. Genet. A 134 (3), 242-246.
Mitsubuchi, H., et al., 2008. Inborn errors of proline metabolism. J. Nutr. 138 (10), 2016S-2020S.
Murphy, K. C., Jones, L. A., Owen, M. J., 1999. High rates of schizophrenia in adults with velo-cardio-facial syndrome. Arch. Gen. Psychiatry 56 (10), 940-945.
Nadler, J. V., 1987. Sodium-dependent proline uptake in the rat hippocampal formation: association with ipsilateral-commissural projections of C A3 pyramidal cells. J. Neurochem. 49 (4), 1155-1160.
Nadler, J. V., Bray, S. D., Evenson, D. A., 1992. Autoradiographic localization of proline uptake in excitatory hippocampal pathways. Hippocampus 2 (3), 269-278.
Nickolson, V. J., 1982. “On” and “off” responses of K+-induced synaptosomal proline release: involvement of the sodium pump. J. Neurochem. 38 (1), 289-292.
Paterlini, M., Zakharenko, S. S., Lai, W. S., Qin, J., Zhang, H., Mukai, J. W., Zakharenko, S. S., Lai, W. S., Qin, J., Zhang, H., Mukai, J. W., et al., 2005.
Transcriptional and behavioral interaction between 22q11.2 orthologs modulates schizophrenia-related phenotypes in mice. Nat. Neurosci. 8 (11), 1586-1594.
Phang, J. M., Hu, C. A., Valle, D., 2001. Disorders of Proline and Hydroxyproline Metabolism, Eighth edition. McGraw-Hill, New York.
Phang J M. et al. (2010) Ann Rev of Nutrit. 30:441-463 Rabinowitz, J., Levine, S. Z., Hafner, H., 2006. A population based elaboration of the role of age of onset on the course of schizophrenia. Schizophr. Res. 88 (1-3), 96-101.
Ramachandran-C et al., ANTICANCER RESEARCH 25: 3293-3302 (2005)
Rao, M. L., Gross, G., Strebel, B., Bräunig, P., Huber, G., Klosterkötter, J., 1990. Serum amino acids, central monoamines, and hormones in drug-naive, drug-free, and neuroleptic-treated schizophrenic patients and healthy subjects. Psychiatry Res. 34, 243-257.
Raux, G., Bumsel, E., Hecketsweiler, B., van Amelsvoort, T., Zinkstok, J., Manouvrier-Hanu, S., et al., 2007. Involvement of hyperprolinemia in cognitive and psychiatric features of the 22q11 deletion syndrome. Hum. Mol. Genet. 16 (1), 83-91.
Renick, S. E., Kleven, D. T., Chan, J., Stenius, K., Milner, T. A., Pickel, V. M., et al., 1999. The mammalian brain high-affinity L-proline transporter is enriched preferentially in synaptic vesicles in a subpopulation of excitatory nerve terminals in rat forebrain. J. Neurosci. 19 (1), 21-33.
Rosen C J. Clinical practice. Vitamin D insufficiency. N Engl J Med 2011; 364(3):248-54.
Scambler, P. J., 2000. The 22q11 deletion syndromes. Hum. Mol. Genet. 9 (16), 2421-2426.
Shafqat, S., Faircloth, M., Henzi, V. A., Whitney, K. D., Yang-Feng, T. L., Seldin, M. F., et al., 1995. Human brain-specific L-proline transporter: molecular cloning, functional expression, and chromosomal localization of the gene in human and mouse genomes. Mol. Pharmacol. 48 (2), 219-229.
Shanti, N. D., Shashikumar, K. C., Desai, P. V., 2004. Influence of proline on rat brain activities of alanine aminotransferase, aspartate aminotransferase and acid phosphatase. Neurochem. Res. 29 (12), 2197-2206.
Tomiya, M., Fukushima, T., Watanabe, H., Fukami, G., Fujisaki, M., Iyo, M., et al., 2007. Alterations in serum amino acid concentrations in male and female schizophrenic patients. Clin. Chim. Acta 380 (1-2), 186-190.
Turnbridge et al., Catechol-o-methyltransferase (COMT) and proline dehydrogenase (PRODH) mRNAs in the dorsolateral prefrontal cortex in schizophrenia, bipolar disorder, and major depression. Synapse. Volume 51, Issue 2, pages 112-118, February 2004
Velaz-Faircloth, M., Guadano-Ferraz, A., Henzi, V. A., Fremeau, R. T., 1995. Mammalian brain-specific L-proline transporter. Neuronal localization of mRNA and enrichment of transporter protein in synaptic plasma membranes. J. Biol. Chem. 270 (26), 15755-15761.
Walter, H., Schlaff, W. B., Lesch, O. M., Vitek, L., Zima, T., Hartl, D., et al., 2008. Breath alcohol level and plasma amino acids: a comparison between older and younger chronic alcohol-dependent patients. Alcohol. 43 (6), 653-657.
Wassef, A. A., Winkler, D. E., Roache, A. L., Abobo, V. B., Lopez, L. M., Averill, J. P., et al., 2005. Lower effectiveness of divalproex versus valproic acid in a prospective, quasiexperimental clinical trial involving 9260 psychiatric admissions. Am. J. Psychiatry 162 (2), 330-339.
Wu, G. Determination of proline by reversed-phase high-performance liquid chromatography with automated pre-column o-phthaldialdehyde derivatization. Journal of Chromatography A. Volume 641, Issue 1, 1993, Pages 168-175.
Yoneda, Y., Roberts, E., 1982. A new synaptosomal biosynthetic pathway of proline from ornithine and its negative feedback inhibition by proline. Brain Res. 239 (2), 479-488.

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

What is claimed is:

1. A method for treating or ameliorating the effects of a schizophrenia-spectrum disorder comprising administering to a patient in need thereof a therapeutically effective amount of a proline modulator.

2. The method according to claim 1, wherein the proline modulator is selected from the group consisting of activators of PRODH and activators of peroxisomal proliferator-activated receptor gamma (PPARγ).

3. The method according to claim 2, wherein the activator of PRODH is vitamin D or an analog thereof.

4. The method according to claim 2, wherein the activator of PRODH is curcumin, or an analog thereof.

5. The method according to claim 2, wherein the activator of PPARγ is selected from the group consisting of troglitazone, rosiglitazone, roglitazone, ciglitazone, darglitazone, englitazone, hydroxypioglitazone, ketopioglitazone, pioglitazone, pioglitazone hydrochloride, rivoglitazone pharmaceutically acceptable salts thereof, and combinations thereof.

6. The method according to claim 1, wherein the schizophrenia-spectrum disorder is schizophrenia.

7. The method according to claim 1, wherein the schizophrenia-spectrum disorder is schizoaffective disorder.

8. The method according to claim 1 further comprising administering to the patient a therapeutically effective amount of an antipsychotic agent, a glutamatergic agent, or a combination thereof.

9. The method according to claim 8, wherein the antipsychotic agent is selected from the group consisting of Haloperidol, Droperidol, Chlorpromazine, Fluphenazine, Perphenazine, Prochlorperazine, Thioridazine, Trifluoperazine, Mesoridazine, Periciazine, Promazine, Triflupromazine, Levomepromazine, Promethazine, Pimozide, Cyamemazine, Chlorprothixene, Clopenthixol, Flupenthixol, Thiothixene, Zuclopenthixol, Clozapine, Olanzapine, Risperidone, Quetiapine, Ziprasidone, Amisulpride, Asenapine, Paliperidone, Iloperidone, Zotepine, Sertindole, Aripiprazole, Cannabidiol, pharmaceutically acceptable salts thereof, and combinations thereof.

10. The method according to claim 8, wherein the glutamatergic agent is selected from the group consisting of D-serine, D-cycloserine, glycine, L-proline, D-aspartate, L-aspartate, L-glutamate, D-glutamate, L-alanine, D-alanine, ketamine, and phencyclidine (pcp), pharmaceutically acceptable salts thereof, and combinations thereof.

11. A method of selecting a patient at risk for or suffering from schizophrenia likely to benefit from proline modulation comprising:
(a) obtaining a biological sample from the patient;
(b) testing the biological sample to determine whether the patient has hyperprolinemia, wherein a patient with hyperprolinemia is a candidate for proline modulation treatment; and
(c) if the patient is determined from step (b) to have hyperprolinemia, administering to the patient an effective amount of an activator of PRODH or an activator of PPARγ.

12. The method according to claim 11, wherein the activator of PRODH is selected from the group consisting of vitamin D, curcumin and an analog thereof.

13. The method according to claim 11, wherein the activator of PPARγ is selected from the group consisting of troglitazone, rosiglitazone, roglitazone, ciglitazone, darglitazone, englitazone, hydroxypioglitazone, ketopioglitazone, pioglitazone, pioglitazone hydrochloride, rivoglitazone pharmaceutically acceptable salts thereof, and combinations thereof.

14. The method according to claim 11, further comprising administering to the patient determined to have hyperprolinemia a therapeutically effective amount of an antipsychotic agent, a glutamatergic agent, or a combination thereof.

15. A composition for treating or ameliorating the effects of schizophrenia comprising an effective amount of a proline modulator, and a pharmaceutically acceptable carrier.

16. The composition according to claim 15, wherein the proline modulator is selected from the group consisting of activators of PRODH and activators of peroxisomal proliferator-activated receptor gamma (PPARγ).

17. The composition according to claim 16, wherein the activator of PRODH is vitamin D or an analog thereof.

18. The composition according to claim 16, wherein the activator of PRODH is curcumin, or an analog thereof.

19. The composition according to claim 16, wherein the activator of PPARγ is selected from the group consisting of troglitazone, rosiglitazone, roglitazone, ciglitazone, darglitazone, englitazone, hydroxypioglitazone, ketopioglitazone, pioglitazone, pioglitazone hydrochloride, rivoglitazone pharmaceutically acceptable salts thereof, and combinations thereof.

20. The composition according to claim 16, further comprising a therapeutically effective amount of an antipsychotic agent, a glutamatergic agent, or a combination thereof.

21. A method for identifying an agent that modulates proline levels in a patient comprising:
(a) administering a candidate agent to a non-human animal having a null mutation of Dtnbp 1;
(b) carrying out an assay to determine whether the candidate agent changes the proline level or the PRODH level in the non-human animal relative to a control;
wherein a candidate agent that causes a change in the proline level or the PRODH level of the non-human animal relative to the control is an agent that modulates proline levels in a patient.

22. A method for identifying whether a patient at risk for developing a DTNBP1-related psychiatric illness or whether a patient having a schizophrenia-spectrum disorder is at risk for an increased length of hospital stay comprising:
(a) obtaining a biological sample from a patient;
(b) carrying out an assay to determine whether the patient has an elevated proline level compared to a control (proline assay) or a decreased PRODH expression level relative to a control (PRODH assay),
wherein a patient with an elevated proline level or a decreased PRODH expression level in step (b) is at risk for developing a DTNBP1-related psychiatric disease and/or is at risk for an increased length of hospital stay.

23. The method according to claim 22, wherein the psychiatric disease is schizophrenia.

24. The method according to claim 22, wherein the biological sample is selected from the group consisting of whole blood, serum, plasma, cerebro-spinal fluid, leukocytes or leukocyte subtype cells, fibroblast sample, and olfactory neuron sample.
